(19) United States
(12) Patent Application Publication
(10) Pub. No.: US 2023/0279000 A1
(43) Pub. Date: Sep. 7, 2023

(54) PYRIDOPYRIMIDINE DERIVATIVES USEFUL IN MODULATION OF AHR SIGNALLING

(71) Applicant: JAGUAHR THERAPEUTICS PTE LTD, Singapore (SG)

(72) Inventors: Mark GRAHAM, Leicestershire (GB); James HITCHIN, Nottingham Nottinghamshire (GB); Sarah MAJOR, Didcot Oxfordshire (GB); Michael STOCKS, Great Dalby Leicestershire (GB); Wendy TOMLINSON, Leicester Leicestershire (GB)

(21) Appl. No.: 17/904,892

(22) PCT Filed: Feb. 26, 2021
(86) PCT No.: PCT/SG2021/050095

§ 371 Date: Aug. 24, 2022

(30) Foreign Application Priority Data
Feb. 26, 2020 (SG) 10202001722X

(51) Int. Cl. C07D 471/04; C07D 487/04

(52) U.S. Cl. CPC C07D 471/04; C07D 487/04

(57) ABSTRACT

The present disclosure relates to compounds of formula (I). Compounds of formula (II), Compounds of formula (III), Compounds of formula (IV), Compounds of formula (V), Compounds of formula (VI), Compounds of formula (VII), in particular inhibitors if AhR, pharmaceutical formulation comprising any one of the same, use of said compounds or compositions in treatment, in particular treatment of cancer.

The present invention relates to compounds of the general formula (I) as described and defined herein, methods for preparing said compounds, pharmaceutical compositions and combinations comprising said compounds and the use of said compounds and pharmaceutical compositions for the treatment or prevention of diseases, in particular cancer or conditions with dysregulated immune functions, or other conditions associated with aberrant AhR signalling, as a sole agent or in combination with other active ingredients. Such compounds may also be of utility in the expansion of hematopoietic stem cells (HSCs) and the use of HSCs in autologous or allogenic transplantation for the treatment of patients with inherited immunological and autoimmune diseases and diverse hematopoietic disorders.

### BACKGROUND

The aryl hydrocarbon receptor (AhR) is a ligand-activated factor that belongs to the family of the basic helix-loop-helix-Per/ARNT/Sim family. Following ligand binding in the cytoplasm, AhR dissociates from its complex with Hsp90 and the AhR-interacting protein, XAP2, allowing ligated AhR to translocate to the nucleus. There, AhR dimerizes with the AhR nuclear translocator (ARNT), that then binds to xenobiotic response elements (XREs) promoting the up- or down-regulation of a multitude of target genes in many different tissues. The AhR is best known for binding to environmental toxins and inducing various members of the cytochrome P450 family including CYP1A1, CYP1A2 and CYP1B1 required for their elimination. Activation of AhR by xenobiotics has demonstrated that this receptor plays a role in a range of physiological processes including embryogenesis, tumourigenesis and inflammation (Esser & Rannug, Pharmacol Rev, 2015, 67:259; Roman et al., Pharmacol Ther, 2018, 185:50).

AhR is expressed in many immune cell types including dendritic cells, macrophages, T cells, NK cells and B cells and plays an important role in immunoregulation (Quintana & Sherr, Pharmacol Rev, 2013, 65:1148; Nguyen et al., Front Immunol, 2014, 5:551). The toxic/adverse effects of classical exogenous AhR agonists, such as 2,3,7,8-Tetrachlorodibenzo-p-dioxin (TCDD) are well known and include profound immunosuppression and initiation of malignancy (Esser et al., Trends Immunol, 2009, 30:447; Feng et al., Biochimica et Biophysica Acta, 2013, 1836:197). Physiological effects of AhR agonists on immune cells include promotion of regulatory T cell (Treg) generation (Pot, Swiss Med Wkly, 2012, 142:w13592), modulation of Th17 cell differentiation and activation (Baricza et al., Cell Mol Life Sci, 2016, 73:95) and stimulation of interleukin-22 (IL-22) expression and/or release from human activated peripheral blood mononuclear cells and T cells (Ramirez et al., Eur J Immunol, 2010, 40:2450; Effner et al., Sci Rep, 2017, 7:44005). AhR also modulates the function of antigen presenting cells, such as dendritic cells and macrophages. AhR activation decreases the expression of class II major histocompatibility complex and co-stimulatory molecules and also the production of Th1 and Th17 polarizing cytokines by dendritic cells (Mezrich et al., J Immunol, 2010, 185:3190; Nguyen et al., Proc Natl Acad Sci USA, 2010, 107:19961; Quintana et al., 2010 Proc Natl Acad Sci USA, 107:20768). Indeed, AhR activation boosts the ability of DCs to promote the differentiation of Tregs (Jurado-Manzano et al., 2017, Immunol Lett, 190:84).

In addition to xenobiotics, the AhR can also bind metabolic products of tryptophan degradation including kynurenine (KYN) and kynurenic acid (KYNA). Indoleamine 2,3 dioxygenase 1 and 2 (ID01/ID02) and tryptophan 2,3-dioxygenase 2 (TD02) catalyse the commitment step of the KYN metabolic pathway and are expressed in immune cells (ID01) and a range of cancer cells (ID01 and TD02)(Pilotte et al., Proc Nat Acad Sci, 2012, 109:2497). Inhibitors of ID01 have attracted much interest as potential new treatments to stimulate the immune system to recognize and eliminate cancer cells (Cheong & Sun, Trends Pharmacol Sci, 2018, 39:307). Traditionally the immunosuppressive effect of ID01 has been attributed mainly to reduced levels of tryptophan, which activates the kinase GCN2 (general control non-derepressible 2) and inhibits T cell proliferation/activation both in tumour draining lymph nodes lymph nodes and in the tumour micro-environment. More recently it has become apparent that some of the efficacy of IDO inhibitors may be the result of decreased production of AhR agonists. These endogenously generated AhR agonists have been shown to elicit a range of effects on immune cells including upregulation of ID01 in dendritic cells (Julliard et al., Front Immunol, 2014, 5:458), inhibition of human T cell proliferation (Frumento et al., J Exp Med, 2002; 196:459; Terness et al., J Exp Med, 2002; 196: 447; Opitz et al., Nature, 2011, 478:197) and up-regulation of PD-1 expression in cytotoxic T lymphocytes (Liu et al., Cancer Cell, 2018; 33:480). As highlighted above, ID01 is not the only source of endogenous AhR agonists. TD02 is predominately expressed in the liver but it is also constitutively expressed in some cancers, notably malignant glioma, hepatocellular carcinoma, melanoma, bladder, breast, lung and colorectal cancer (Opitz et al., Nature, 2011, 478:197; Pilotte et al., Proc Nat Acad Sci, 2012, 109:2497; D’Amato et al., Cancer Res, 2015, 75(21):4651; Hsu et al., Oncotarget, 2016, 7(19): 27584; Chen et al., Dis Markers, 2016, 2016:8169724). Such data suggests that AhR antagonists may have broader efficacy than selective IDO-1 inhibitors, as they will attenuate endogenous AhR agonist signalling regardless of its source. This assertion was given more weight by the recent discovery of another enzyme, Interleukin-4 induced 1 (IL4I1), capable of generating endogenous AhR agonists (Sadik et al., Cell, 2020, 182:10).

In addition to their effects on immune cells, such endogenous agonists have also been implicated in cancer progression via direct effects on the tumour. For example, KYN increases human glioblastoma cell survival and migration (Opitz et al., Nature, 2011, 478:197). Several other studies also implicate the AhR in cancer progression in the absence of environmental ligands. The AhR-repressor (AHRR) protein acts as a tumour suppressor gene in several human cancers (Zudaire et al., J Clin Invest, 2008, 118:640). AhR expression and “constitutive” (endogenous ligand-driven) activity in breast cancer cells correlate with tumour aggressiveness (Schlezinger et al., Biol Chem, 2006, 387:1175; Yang et al., J Cell Biochem, 2008, 104:402) and control expression of genes associated with tumour invasion (Yang et al., Oncogene, 2005, 24:7869). Ectopic AhR expression in non-malignant human mammary epithelial cells induces an epithelial-to-mesenchymal transition and a > 50% increase in cell growth rates (Brooks & Eltom, Curr Cancer Drug Targets, 2011, 11:654) and AhR knockdown induced gene changes in human breast cancer cell lines consistent with a mesenchymal to epithelial cell reversion to a less aggressive phenotype (Narasimhan et al., Int J Mol Sci, 2018, 19:1388). AhR antagonists or AhR knockdown has been shown to reduce proliferation, survival, invasiveness and migration of human breast cancer cells in culture (Parks et al., Mol Pharmacol, 2014, 86:593; D’Amato et al., Cancer Res, 2015, 75(21):4651; Narasimhan et al., Int J Mol Sci, 2018, 19:1388) and to reduce survival of glioblastoma cells (Gramatzki et al., Oncogene, 2009, 28:2593; Opitz et al., Nature, 2011, 478:197; Guastella et al., J Neuro-oncol, 2018, in press). Finally, AhR antagonists block the formation of tumourspheres (Stanford et al., Mol Cancer Res, 2016, 14:696) which are formed by cancer stem cells (CSCs), a subset of tumour cells that drive the initiation, progression and metastasis of tumours.

Thus, AhR agonists released from immune cells and from tumour cells act in an autocrine and paracrine fashion to promote tumour growth. Agents that reduce or block these effects may therefore find utility in the treatment of cancer and/or conditions with dysregulated immune functions. Thus such agents may also have utility in a range of other diseases/conditions including but not limited to, obesity (Rojas et al., Int J Obesity, 2020, 44:948) and various viral infections (Giovannoni et al., Nat Neurosci. 2020, 23:939; Giovannoni et al., Res Sq. 2020, rs.3.rs-25639).

WO2017/202816 relates to compounds and compositions for the treatment or prophylaxis of cancer or conditions with dysregulated immune responses or other disorders associated with aberrant AhR signalling. In particular, WO2017/202816 WO2018/146010 and WO2019/101642 relate inter alia to heterocyclic compounds capable of inhibiting AhR function.

WO2020/081840 relates to aryl hydrocarbon receptor antagonists, such as substituted imidazopyridines and imidazopyrazines, as well as methods of expanding hematopoietic stem cells by culturing hematopoietic stem or progenitor cells in the presence of these agents.

WO2020/039093 relates to compositions and methods for using tetrahydropyridopyrimidine derivatives as AhR modulators.

WO2018/153893 relates to 6-amido-1H-indol-2-yl compounds which can act as aryl hydrocarbon receptor (AhR) modulators and, in particular, as AhR antagonists. The invention further relates to the use of the compounds for the treatment and/or prophylaxis of diseases and/or conditions through binding of said aryl hydrocarbon receptor by said compounds.

WO2020/021024 relates to bicyclic compounds which can act as aryl hydrocarbon receptor (AhR) modulators and, in particular, as AhR antagonists. The invention further relates to the use of the compounds for the treatment and/or prophylaxis of diseases and/or conditions through binding of said aryl hydrocarbon receptor by said compounds.

WO2020/043880 relates to heterocyclic compounds which are ARH inhibitors, for prevention of diseases, in particular cancer or conditions with dysregulated immune functions, or other conditions associated with aberrant AHR signalling, as a sole agent of in combination with other active ingredients.

WO 2020/018848 relates to methods for expanding stem cells and/or lineage committed progenitor cells, such as hematopoietic stems cells and/or lineage committed progenitor cells, at least in part, by using compounds that antagonize AhR.

WO2020/050409 relates to novel heterocyclic compound having an aryl hydrocarbon receptor antagonist activity and useful for the promotion of platelet production.

WO 2019/236766 relates to methods for expanding stem cells and/or lineage committed progenitor cells, at least in part, by using lactam compounds that antagonize AhR.

WO2019/018562 relates to compositions and methods of using heteroaryl amides as AhR modulator compounds, for the treatment of diseases modulated, as least in part, by AhR.

WO 2018/195397 relates to compositions and methods for indole AhR inhibitors.

WO 2018/146010 relates to the preparation of 2-heteroaryl-3-oxo-2,3-dihydropyridazine-4-carboxamides for the treatment or prophylaxis of diseases, in particular cancer or conditions with dysregulated immune responses, as a sole agent or in combination with other active ingredients.

WO2010/059401 relates to compounds and compositions for expanding the number of CD34+ cells for transplantation. In particular, WO 2010/059401 relates inter alia to heterocyclic compounds capable of down regulating the activity and/or expression of AhR.

WO2012/015914 relates to compositions and methods for modulating AhR activity. In particular, WO2012/015914 relates inter alia to heterocyclic compounds that modulate AhR activity for use in therapeutic compositions to inhibit cancer cell proliferation and tumour cell invasion and metastasis.

WO2020/051207 relates to AhR antagonists as well as methods of modulating AhR activity and expanding hematopoietic stem cells by culturing hematopoietic stem or progenitor cells in the presence of these agents. Additionally, this disclosure provides methods of treating various pathologies, such as cancer, by administration of these AhR antagonists

US2018/327411 A1 relates to compounds and compositions useful as inhibitors of AhR to treat a variety of diseases, disorders and conditions associated with AhR.

US2019/389857 A1 relates to compounds which can act as AhR modulators, and in particular, as AhR antagonists.

### SUMMARY OF THE DISCLOSURE

The present disclosure provides pyrimidine compounds of general formula (I) which inhibit the AhR. The disclosure is summarised in the following paragraphs:
1. A compound of formula (I)

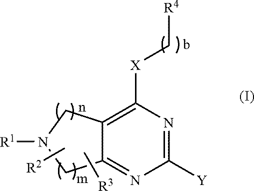

wherein:
   Y is a 5 or 6 membered ring optionally comprising 1, 2, or 3 heteroatoms selected from N, O and S, substituted with R⁵ and R⁶;
   R¹ is H, C₁₋₃ alkyl, (—CH₂)pCN, -COC₁₋₃ alkyl, —CO(CH₂)qNR⁷R⁸, -SO₂C₁₋₃ alkyl, —SO₂NR⁷R⁸, —(CH₂)qPh, —C(O)Z;
   R² is H or C₁₋₃ alkyl;
   R³ is H or C₁₋₃ alkyl;
   R⁴ is a 9 or 10 membered heteroaryl with at least one heteroatom selected from N, O or S (such as Indol-3-yl or Benzimidazol-2-yl), with substituents R⁹ and R¹⁰;
   R⁵ is H, hydroxy, halogen (such as F, Cl), CN, C₁₋₃ alkyl, C₁₋₃ alkoxy (such as OMe), C₁₋₂ haloalkyl (such as CF₃), C₁₋₃ alkyl(OH), —CO(CH₂)qNR⁷R⁸, —SO₂C₁₋₃ alkyl, —SO₂ NR⁷R⁸,
   R⁶ is H, hydroxy, halogen (such as F, Cl), CN, C₁₋₃ alkyl, C₁₋₃ alkoxy (such as OMe), C₁₋₂ haloalkyl (such as CF₃), C₁₋₃ alkyl(OH), —CO(CH₂)qNR⁷R⁸, —SO₂C₁₋₃ alkyl, —SO₂ NR⁷R⁸ (such as H, hydroxy, halogen (such as F, Cl), CN, C₁₋₃ alkyl, —CO(CH₂)qNR⁷R⁸, —SO₂C₁₋₃ alkyl, —SO₂ NR⁷R⁸),
   R⁷ is H or C₁₋₃ alkyl, such as —CH₃;
   R⁸ is H or C₁₋₃ alkyl, such as —CH₃;
   R⁹ is H, hydroxy, halogen (such as F, Cl), CN, C₁₋₃ alkyl, C₁₋₃ alkoxy (such as OMe), C₁₋₂ haloalkyl (such as CF₃), C₁₋₃ alkyl(OH), —CO(CH₂)q NR⁷R⁸, —SO₂C₁₋₃ alkyl, or —SO₂ NR⁷R⁸,
   R¹⁰ is H, hydroxy, halogen (such as F, Cl), CN, C₁₋₃ alkyl, C₁₋₃ alkoxy (such as OMe), C₁₋₂ haloalkyl (such as CF₃), C₁₋₃ alkyl(OH), —CO(CH₂)q NR⁷R⁸, —SO₂C₁₋₃ alkyl, or —SO₂ NR⁷R⁸ (such as H, hydroxy, halogen (such as F, Cl), CN, C₁₋₃ alkyl, —CO(CH₂)q NR⁷R⁸, —SO₂C₁₋₃ alkyl, or —SO₂ NR⁷R⁸),
   R¹¹ is H or C₁₋₃ alkyl (such as —CH₃);
   X is CH₂, S, —SO₂, NR¹¹ or O;
   Z is a 5 or 6 membered heteroaryl with at least one heteroatom selected from N, O and S, for example 1 or 2 nitrogens, wherein said heteroaryl optionally bears one or two substituents selected from hydroxy, halogen (such as F, Cl), CN, C₁₋₃ alkyl;
   b is 0, 1, 2 or 3 (for example 0 or 2);
   n is an integer 1 or 2;
   m is an integer 1 or 2;
   p is an integer 1, 2 or 3 (such as 1);
   q is 0, 1, 2 or 3 (such as 0 or 1),
   or a pharmaceutically acceptable salt thereof
   with the proviso that when X is NR¹¹ or O and b is 1 or 2 then R⁵ or R⁹ is selected from C₁₋₃
   alkoxy (such as OMe), C₁₋₂ haloalkyl (such as CF₃), and C₁₋₃ alkyl(OH).
2. A compound of formula (I) wherein Y is a 5 or 6 membered nitrogen containing ring.
3. A compound of formula (I) according to claim 2, wherein the ring is aromatic.
4. A compound of formula (I) according to claim 3, wherein the ring is pyrimidine or pyridine.
5. A compound of formula (I) according to claim 4, wherein R⁵ is located at position 5.
6. A compound of formula (II)

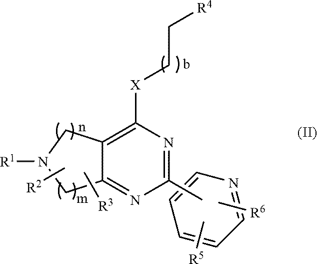

wherein X, R¹, R², R³, R⁴, R⁵, R⁶, b, m and n are defined above for compounds of formula (I) or a pharmaceutically acceptable salt thereof.
7. A compound of formula (III):

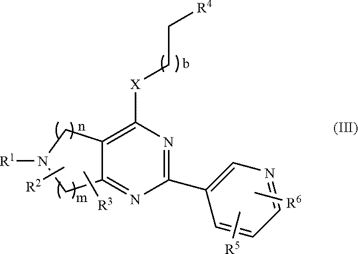

wherein X, R¹, R², R³, R⁴, R⁵, R⁶, b, m and n are defined above for compounds of formula (I) or a pharmaceutically acceptable salt thereof.
8. A compound according to any one of paragraphs 1 to 7 wherein n is 2.
9. A compound according to any one of paragraphs 1 to 7, wherein n is 1.
10. A compound according to any one of paragraphs 1 to 9, wherein m is 2.
11. A compound according to any one of paragraphs 1 to 9, wherein m is 1.
12. A compound according to any one of paragraphs 1 to 7, of formula (IV):

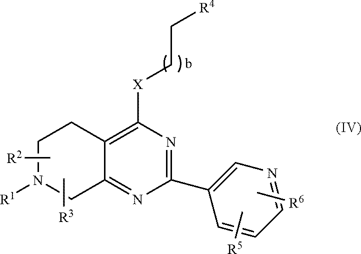

wherein X, R¹, R², R³, R⁴, R⁵, R⁶ and b, are defined above for compounds of formula (I) or a pharmaceutically acceptable salt thereof.
13. A compound according to any one of paragraphs 1 to 7, of formula (V):

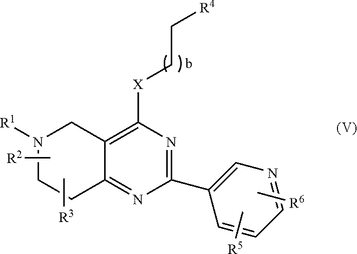

wherein X, R¹, R², R³, R⁴, R⁵, R⁶ and b, are defined above for compounds of formula (I) or a pharmaceutically acceptable salt thereof.
14 A compound according to any one of paragraphs 1 to 7, of formula (VI):

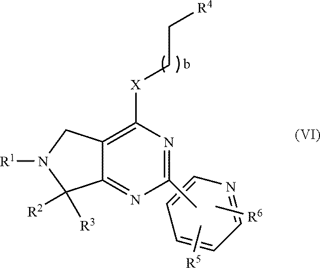

wherein X, R¹, R², R³, R⁴, R⁵, R⁶ and b, are defined above for compounds of formula (I) or a pharmaceutically acceptable salt thereof.
15. A compound according to any one of paragraphs 1 to 7, of formula (VII):

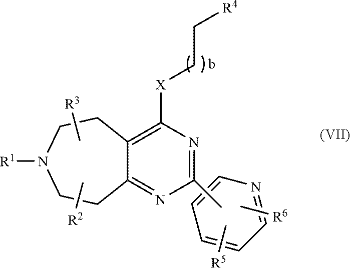

wherein X, R¹, R², R³, R⁴, R⁵, R⁶ and b, are defined above for compounds of formula (I) or a pharmaceutically acceptable salt thereof.
16. A compound according to any one of paragraphs 1 to 15, wherein R¹ is independently selected from H, CH₃, —CH₂CH₃, —CH₂CH₂CH₃, —CH(CH₃)₂, —C(O)CH₃, C(O)NH₂, —C(O)NHCH₃. —C(O)N(CH₃)₂, —CH₂CN, —SO₂NH₂, —SO₂CH₃, —SO₂N(CH₃)₂, —CH₂Ph, -C(O)1-Me-Pyrazol-5-yl.
17. A compound according to any one of paragraphs 1 to 15, wherein R¹ is independently selected from H, CH₃, —CH₂CH₃, —CH₂CH₂CH₃, —CH(CH₃)₂, —C(O)NH₂, —C(O)NHCH₃. —C(O)N(CH₃)₂, —CH₂CN, —SO₂NH₂, —SO₂CH₃, —SO₂N(CH₃)₂, —CH₂Ph, -C(O)1-Me-Pyrazol-5-yl.
18. A compound according to paragraph 17, wherein R¹ is selected from H, —CH₂CN, —SO₂CH₃, and —SO₂N(CH₃)₂, —C(O)N(CH₃)₂.
19. A compound according to paragraphs 17 or 18, wherein the R¹ is H.
20. A compound according to any one of paragraphs 1 to 15, wherein R¹ is C₁₋₃ alkyl, such as —CH₂CH₃.
21. A compound according to any one of paragraphs 1 to 20, wherein R² is H or —CH₃.
22. A compound according to claim 21, wherein R² is H.
23. A compound according to any one of paragraphs 1 to 22, wherein R³ is H or —CH₃.
24. A compound according to paragraph 23, wherein R³ is H.
25. A compound according to any one of paragraphs 1 to 24, wherein R⁴is selected from indolyl (such as indol-3-yl, in particular 5-fluoro-1H-indol-3-yl) and benzimidazolyl (such as benzimidazole-2-yl), each independently bearing R⁹ and R¹⁰.
26. A compound according to any one of paragraphs 1 to 24, wherein R⁵ is selected from hydroxy, halogen (such as F, Cl), CN, C₁₋₃ alkyl, C₁₋₃ alkoxy (such as OMe), C₁₋₂ haloalkyl (such as CF₃), C₁₋₃ alkyl(OH), —CO(CH₂)qNR⁷R⁸, -SO₂C₁₋₃alkyl, —SO₂ NR⁷R⁸
27. A compound according to any one of paragraphs 1 to 26, wherein R⁵ is selected from H, F, Cl, —CF₃, —SO₂CH₃, —CH₂CH₂OH, CN, —OCH₃ and —CH₃.
28. A compound according to any one of paragraphs 1 to 25, wherein R⁵ is H.
29. A compound according to any one of paragraphs 1 to 25, wherein R⁵ is F.
30. A compound according to any one of paragraphs 1to 25, wherein R⁵ is Cl.
31. A compound according to any one of paragraphs 1 to 25, wherein R⁵ is —CF₃.
32. A compound according to any one of paragraphs 1to 25, wherein R⁵ is —SO₂CH₃.
33. A compound according to any one of paragraphs 1 to 25, wherein R⁵ is —CH₂CH₂OH.
34. A compound according to any one of paragraphs 1 to 25, wherein R⁵ is CN.
35. A compound according to any one of paragraphs 1 to 25, wherein R⁵ is —OCH₃.
36. A compound according to any one of paragraphs 1 to 25, wherein R⁵ is —CH₃.
37. A compound according to any one of paragraphs 1 to 36, wherein R⁵ is beta to the atom which is bonded to the remainder of the molecule.
38. A compound according to any one of paragraphs 1 to 37, wherein R⁶ is H, F, Cl, CN or —CH₃.
39. A compound according to paragraph 38, wherein R⁶ is H.
40. A compound according to any one of paragraphs 1 to 39, wherein R⁷ is selected from H and —CH₃.
41. A compound according to paragraph 40, wherein R⁷ is —CH₃.
42. A compound according to paragraph 40, wherein R⁷ is H.
43. A compound according to any one of paragraphs 1 to 42, wherein R⁸ is selected from H and —CH₃.
44. A compound according to paragraph 43, wherein R⁸ is H.
45. A compound according to paragraph 43, wherein R⁸ is —CH₃.
46. A compound according to any one of paragraphs 1 to 45, wherein R⁹ is selected from H, F, Cl, —CF₃, —SO₂CH₃, —CH₂CH₂OH, CN, —OCH₃ and —CH₃.
47. A compound according to paragraph 46, wherein R⁹ is H.
48. A compound according to paragraph 46, wherein R⁹ is F.
49. A compound according to paragraph 46, wherein R⁹ is Cl.
50. A compound according to paragraph 46, wherein R⁹ is CF₃.
51. A compound according to paragraph 46, wherein R⁹ is —SO₂CH₃.
52. A compound according to paragraph 46, wherein R⁹ is —CH₂CH₂OH.
53. A compound according to paragraph 46, wherein R⁹ is CN.
54. A compound according to paragraph 46, wherein R⁹ is —OCH₃.
55. A compound according to paragraph 46, wherein R⁹ is —CH₃.
56. A compound according to any one of paragraph 1 to 55, wherein R¹⁰ is H.
57. A compound according to any one of paragraphs 1 to 56, wherein R¹⁰ is methyl.
58. A compound according to any one of paragraphs 1 to 57, wherein R¹¹ is H.
59. A compound according to any one of paragraphs 1 to 58, wherein b is 0.
60. A compound according to any one of paragraphs 1 to 58, wherein b is 1.
61. A compound according to any one of paragraphs 1 to 58, wherein b is 2.
62. A compound according to any one of paragraphs 1 to 58, wherein b is 3.
63. A compound according to any one of paragraphs 1 to 62, wherein p is 1.
64. A compound according to any one of paragraphs 1 to 63, wherein q is 1.
65. A compound according to any one of paragraphs 1 to 63, wherein q is 0.
66. A compound according to any one of paragraphs 1 to 65, wherein X is CH₂
67. A compound according to any one of paragraphs 1 to 65, wherein X is S.
68. A compound according to any one of paragraphs 1 to 65, wherein X is NR¹¹.
69. A compound according to any one of paragraphs 1 to 65, wherein X is O.
70. A pharmaceutical composition comprising a compound according to any one of paragraphs 1 to 69 and an excipient, diluent or carrier.
71. A compound according to any one of claims 1 to 69 or a pharmaceutical composition according to paragraph 70, for use in treatment.
72. A compound or composition for use according to paragraph 71, for use in the treatment of cancer.
73. A method of treating a patient comprising administering a therapeutically effective amount of a compound as defined in any one of paragraphs 1 to 69 or a composition as defined in paragraph 70.
74. Use of a compound according to any one of paragraphs 1 to 69 or a composition according to paragraph 70, for the manufacture of a medicament for the treatment of cancer.

In one embodiment m is 1 and n is 1. In one embodiment m is 1 and n is 2. In one embodiment m is 2 and n is 1. In one embodiment m is 2 and n is 2. In one embodiment n is 1 and m is 1 or 2.

In one embodiment R⁴ is indole, for example substituted according to the present disclosure, such as substituted at a position selected from 2; 2 and 5; and 6.

In one embodiment R⁴ is 2-trifluoromethyl-1H indoly-3-yl.

In one embodiment R⁴ is 6-methoxy-1H indoly-3-yl.

In one embodiment R⁴ is 5-methoxy, 2-methyl-1H indoly-3-yl.

In one embodiment Y is pyrimidine, including pyrimidine substituted by R⁵ and R⁶, for example wherein at least one of R⁵ or R⁶ is not H.

In one embodiment Z is unsubstituted.

In one embodiment X is O or NH, Y is pyridyl or pyrimidinyl, R¹ is H or ethyl, R² is H, R³, is H, R⁴ is indolyl (such as indol-3yl, in particular substituted at positions 2, 2+5, and 6), R⁵ is F, —OCH₃ or CF₃ (for example in position 5), R⁶ is H, R⁹ is H, F and OCH₃, including a pharmaceutically acceptable salt thereof.

The present disclosure also includes individual compounds disclosed herein, pharmaceutical salts thereof, pharmaceutical formulations thereof and therapeutic uses of anyone of the same, in particular as described elsewhere herein.

In one embodiment the disclosure provides examples 1, 2, 3, 4, 5, 6, 7, 8, 9, 10, 11, 12, 13, 14, 15, 16, 17 and 18, a pharmaceutical salt of any one of the same and/or a pharmaceutical formulation of any one of the same.

In one embodiment the salt is selected from chloride and hydrochloride.

In one embodiment the disclosure provides: 2-(5-fluoropyridin-3-yl)-N-[2-(6-methoxy-1H-indol-3-yl)ethyl]-5H,6H,7H,8H-pyrido[3,4-d]pyrimidin-4-amine); 2-(5-fluoropyridin-3-yl)-N-[2-(6-methoxy-1H-indol-3-yl)ethyl]-5H,6H,7H-pyrrolo[3,4-d]pyrimidin-4-amine; N-[2-(1H-indol-3-yl)ethyl]-2-(5-methoxypyridin-3-yl)-5H,6H,7H,8H-pyrido[3,4-d]pyrimidin-4-amine); N-[2-(1H-indol-3-yl)ethyl]-2-[5-(trifluoromethyl)pyridin-3-yl]-5H,6H,7H,8H-pyrido[3,4-d]pyrimidin-4-amine); 2-(5-fluoropyridin-3-yl)-4-{[2-(5-methoxy-2-methyl-1H-indol-3-yl)ethyl]amino}-5H,6H,7H,8H-pyrido[3,4-d]pyrimidin-7-ium [such as chloride]; 2-(5-fluoropyridin-3-yl)-N-[2-(6-methoxy-1H-indol-3-yl)ethyl]-5H,6H,7H,8H,9H-pyrimido[4,5-d]azepin-4-amine; 7-ethyl-2-(5-fluoropyridin-3-yl)-N-[2-(6-methoxy-1H-indol-3-yl)ethyl]-5H,6H,7H,8H,9H-pyrimido[4,5-d]azepin-4-amine; 3-(2-{[2-(5-fluoropyridin-3-yl)-5H,6H,7H,8H-pyrido[3,4-d]pyrimidin-4-yl]oxy}ethyl)-6-methoxy-1H-indole [such as the hydrochloride; 2-(5-fluoropyridin-3-yl)-4-({2-[2-(trifluoromethyl)-1H-indol-3-yl]ethyl}amino)-5H,6H,7H,8H-pyrido[3,4-d]pyrimidin-7-ium [such as chloride]; a pharmaceutical salt of any one of the same and/or a pharmaceutical formulation of any one of the same.

The compounds of the present disclosure extend to forms comprising atoms which are “rare” isotopes of more commonly occurring forms of the element, for example deuterium.

In one embodiment the compounds of the present disclosure are AhR inhibitors.

The compounds of the present disclosure include radiolabelled forms thereof.

In particular, the compounds of the present invention have surprisingly been found to effectively inhibit AhR. Said compounds are useful for the treatment or prophylaxis of conditions where exogenous and endogenous AhR ligands induce dysregulated immune responses, for example: uncontrolled cell growth, proliferation and/or survival of tumour cells, immunosuppression. This dysregulation may be observed in the context of cancer, inappropriate cellular immune responses, and inappropriate cellular inflammatory responses.

In one embodiment the compounds of the present disclosure are useful in the treatment of cancer for example, liquid and/or solid tumours, and/or metastases thereof. Examples of cancers include head and neck cancer (such as brain tumours and brain metastases), cancer of the thorax including non-small cell and small cell lung cancer, gastrointestinal cancer (including stomach, oesophageal, colon, and colorectal), biliary tract cancer, pancreatic cancer, liver cancer, endocrine cancer, breast cancer, ovarian cancer, bladder cancer, kidney cancer, prostate cancer, bone cancer and skin cancer.

In one embodiment the cancer is an epithelial cancer. In one embodiment the cancer is a sarcoma. In one embodiment the cancer is a metastatic.

### DETAILED DISCLOSURE

A 5 or 6 membered ring as optionally comprising 1, 2 or 3 heteroatoms selected from nitrogen, oxygen and sulfur, refers to a saturated, partially saturated or aromatic ring containing 5 or 6 atoms, including wherein all the atoms are carbon or where there are 1, 2 or 3 heteroatoms independently selected from nitrogen, oxygen and sulfur, for example cyclopentadiene, phenyl, thiophene, furan, pyrrole, pyrazole, imidazole, oxazole, thiazole, isothiazole, triazole, pyridine, pyrazine, triazine, thiazine, oxazine, cyclopentane, cyclohexane, pyrrolidine, pyrroline, pyrazolidine, imidazoline, tetrahydrofuran, tetrahydrothiophene, piperidine, piperazine, tetrahydrapyran, thiane, thiopyran, morpholine, or thiomorpholine.

In one embodiment the ring is 5 membered.

In one embodiment the ring is 6 membered.

In one embodiment the 5 or 6 membered ring is unsaturated or aromatic (i.e. a 5 or 6 membered heteroaryl).

5 or 6 membered heteroaryl as employed herein is a ring containing 5 or 6 atoms wherein at least one atom is a heteroatom, for example selected from nitrogen, oxygen or sulphur, such as pyrrole, pyrazole, imidazole, thiophene, oxazole, isothiazole, thiazole, pyridine, pyridazine, pyrazine, triazine, thiopyran, oxazine and thiazine, such as pyrrole, pyrazole and pyridine and pyrimidine.

In one embodiment the 5 or 6 membered ring is selected from cyclopentadiene, phenyl, pyridine and pyrazine, such as phenyl and pyridine.

Thus, in one embodiment Y is pyridine or pyrimidine (i.e. includes substituted with R⁵ and R⁶).

In one embodiment Y is pyrazole.

In one embodiment Y is not pyrazole.

C₁₋₃ alkyl as employed herein refers to straight or branched chain alkyl, for example methyl, ethyl, propyl or isopropyl.

Halogen as employed herein includes fluoro, chloro, bromo or iodo.

CO represents carbonyl.

9 or 10 membered heteroaryl as employed herein refers to a bicyclic ring system containing 9 or 10 atoms, wherein at least one ring is aromatic and at least one ring contains a heteroatom, for example containing 1, 2, 3, or 4 heteroatoms independently selected from nitrogen, oxygen and sulfur, such as indoline, indole, isoindole, indolizine, indazole, benzimidazole, azaindole, pyrazolopyrimidine, purine, benzofuran, isobenzofuran, benzothiophene, benzoisooxazole, benzoisothiazole, benzoxazole, benzothiadiazole, adenine, guanine, tetrahydroquinoline, dihydroisoquinoline, quinoline, isoquinoline, quinolizine, quinoxaline, phthalazine, cinnoline, napthrhyridine, pyridopyrimidine, pyridopyrazine, pyridopyrazine, pteridine, chromene, isochromene, chromenone, benzoxazine, quinolinone, and isoquinolinone.

In one embodiment the 9 or 10 membered heteroaryl is selected from indolylyl and benzimidazolyl, such as indol-3-yl or benzimidazole-2-yl.

The compounds of the present disclosure can be prepared by methods described herein. GENERIC ROUTE 1 can be employed to make certain compounds of the present disclosure:

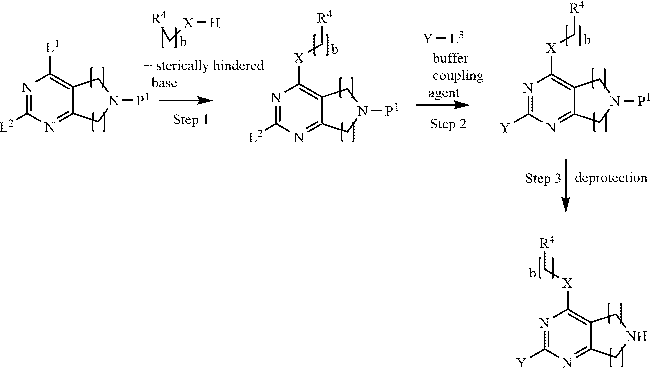

wherein
L¹ and L² are leaving groups, for example halogen, such as chlorine;
L³ is a leaving group, for example boronic acid;
P¹ is a protecting group, for example Boc;
X is NR¹¹, O or S; and
R⁴ and Y are defined above for compounds of formula (I).

GENERIC ROUTE 2 can be employed to make certain compounds of the present disclosure:

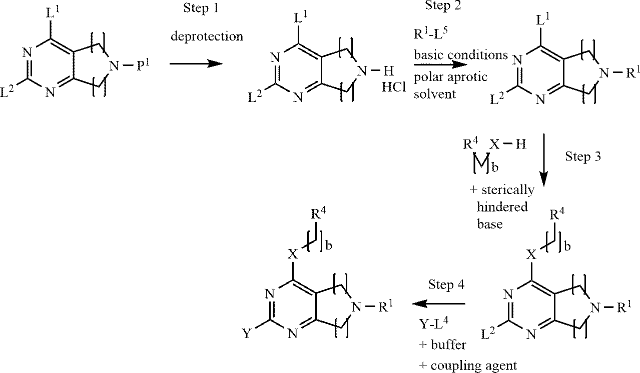

L¹ and L² are leaving groups, for example halogen, such as chloro;
L³ is a leaving group, for example boronic acid;
L⁴ is a leaving groups, for example halogen, such as bromo;
P¹ is a protecting group, for example Boc;
X is NR¹¹, O or S; and
R¹, R⁴ and Y are defined above for compounds of formula (I).

Compounds of formula (I) wherein X is CH₂ can be prepared through palladium catalysed reactions.

An example of a sterically hindered base is triethylamine, which may be employed in step 1 of scheme 1 and step 3 of scheme 2 with tryptamine.

A suitable buffer in step 2 of scheme 1 is aryl boronic acid and potassium carbonate, for example in a solvent, such as dioxan and water.

Coupling agents may require performing the reaction under nitrogen. Suitable coupling agents in for step 2 of scheme 1 and step 4 of scheme 2 include bis(diphenylphosphino) ferrocene]dichloropalladium (II) dichlorine.

Deprotection in step 3 of scheme 1 and step 1 of scheme 2 may be effected using, for example TFA, in particular in dichloromethane.

Step 2 of scheme 2 may be performed in the presence of a sterically hindered organic base, such as a triethylamine. A suitable polar aprotic solvent for the reaction is dichloromethane.

Protecting groups may be required to protect chemically sensitive groups during one or more of the reactions described above, to ensure that the process is efficient. Thus, if desired or necessary, intermediate compounds may be protected by the use of conventional protecting groups. Protecting groups and means for their removal are described in “Protective Groups in Organic Synthesis”, by Theodora W. Greene and Peter G.M. Wuts, published by John Wiley & Sons Inc; 4^{th} Rev Ed., 2006, ISBN-10: 0471697540.

Examples of salts of compound of the present disclosure include all pharmaceutically acceptable salts, such as, without limitation, acid addition salts of strong mineral acids such as HCl and HBr salts and addition salts of strong organic acids, such as a methansulfonic acid salt.

The present disclosure extends to solvates of the compounds disclosed herein. Examples of solvates include hydrates.

Novel intermediates are also an aspect of the invention.

A further aspect of the present disclosure is methods of making the compounds and/or intermediates disclosed herein.

Also provided herein a pharmaceutically composition comprising a compound according to the present disclosure and an excipient, diluent or carrier. A thorough discussion of pharmaceutically acceptable carriers is available in Remington’s Pharmaceutical Sciences (Mack Publishing Company, N.J. 1991).

The pharmaceutical compositions of this disclosure may be administered by any number of routes including, but not limited to, oral, intravenous, intramuscular, intra-arterial, intramedullary, intrathecal, intraventricular, transdermal, transcutaneous (for example, see WO98/20734), subcutaneous, intraperitoneal, intranasal, enteral, topical, sublingual, intravaginal or rectal routes. Hyposprays may also be used to administer the pharmaceutical compositions of the invention.

In one embodiment the therapeutic compositions may be prepared as injectables, either as liquid solutions or suspensions. Solid forms suitable for solution in, or suspension in, liquid vehicles prior to injection may also be prepared. Suitable liquids for reconstitution of such solid forms (including lyophilised solids) may be selected from aqueous solutions, for example saline, dextrose or water for injection and the like. In one embodiment the reconstituted liquid formulation is isotonic.

In one embodiment the pharmaceutical composition according to the present disclosure is provided as a tablet or a capsule for oral administration.

### TREATMENT

The present disclosure also extends to methods of treating a patient comprising administering a therapeutically effective amount of a compound of the present disclosure (or a pharmaceutical composition comprising the same), for example for the treatment of cancer.

Also provide is a compound according to the present disclosure (or a pharmaceutical composition comprising the same) for use in treatment, for example for use in the treatment of cancer.

In a further aspect there is provided a compound of the present disclosure (or a pharmaceutical composition comprising the same) for use in the manufacture of a medicament for the treatment of cancer.

In one embodiment the cancer is an epithelial cancer, for example selected from example is selected from liver cancer (such as hepatocellular carcinoma), biliary tract cancer, breast cancer (such as none ER+ breast cancer), prostate cancer, colorectal cancer, ovarian cancer, cervical cancer, lung cancer, gastric cancer, pancreatic, bone cancer, bladder cancer, head and neck cancer, thyroid cancer, skin cancer, renal cancer, and oesophagus cancer, for example gastric cancer.

In one embodiment the cancer is selected from selected from the group comprising hepatocellular carcinoma, cholangiocarcinoma, breast cancer, prostate cancer, colorecetal cancer, ovarian cancer, lung cancer, gastric cancer, pancreatic and oesophagus cancer.

In one embodiment the biliary duct cancer is in a location selected from intrahepatic bile ducts, left hepatic duct, right hepatic duct, common hepatic duct, cystic duct, common bile duct, Ampulla of Vater and combinations thereof.

In one embodiment the biliary duct cancer is in an intrahepatic bile duct. In one embodiment the biliary duct cancer is in a left hepatic duct. In one embodiment the biliary duct cancer is in a right hepatic duct. In one embodiment the biliary duct cancer is in a common hepatic duct. In one embodiment the biliary duct cancer is in a cystic duct. In one embodiment the biliary duct cancer is in a common bile duct. In one embodiment the biliary duct cancer is in an Ampulla of Vater. In one embodiment the epithelial cancer is a carcinoma.

In one embodiment the treatment according to the disclosure is adjuvant therapy, for example after surgery.

In one embodiment the therapy according to the disclosure is neoadjuvant treatment, for example to shrink a tumour before surgery.

In one embodiment the tumour is a solid tumour. In one embodiment the cancer is a primary cancer, secondary cancer, metastasis or combination thereof. In one embodiment the treatment according to the present disclosure is suitable for the treatment of secondary tumours. In one embodiment the cancer is metastatic cancer. In one embodiment the treatment according to the present disclosure is suitable for the treatment of primary cancer and metastases. In one embodiment the treatment according to the present disclosure is suitable for the treatment of secondary cancer and metastases. In one embodiment the treatment according to the present disclosure is suitable for the treatment of primary cancer, secondary cancer and metastases.

In one embodiment the treatment according to the present disclosure is suitable for the treatment of cancerous cells in a lymph node.

In one embodiment the liver cancer is primary liver cancer. In one embodiment the liver cancer is secondary liver cancer. In one embodiment the liver cancer is stage 1, 2, 3A, 3B, 3C, 4A or 4B.

In one embodiment the gastric cancer is stage 0, I, II, III or IV.

The precise therapeutically effective amount for a human subject will depend upon the severity of the disease state, the general health of the subject, the age, weight and gender of the subject, diet, time and frequency of administration, drug combination(s), reaction sensitivities and tolerance/response to therapy. This amount can be determined by routine experimentation and is within the judgement of the clinician. Generally, a therapeutically effective amount will be from 0.01 mg/kg to 1000 mg/kg, for example 0.1 mg/kg to 500 mg/kg. Pharmaceutical compositions may be conveniently presented in unit dose forms containing a predetermined amount of an active agent of the invention per dose.

### Combination Therapy

In one embodiment the compound of the present disclosure is employed in combination therapy, for example wherein the further therapy is an anticancer therapy.

In one embodiment the anticancer therapy is a chemotherapy.

Chemotherapeutic agent and chemotherapy or cytotoxic agent are employed interchangeably herein unless the context indicates otherwise.

Chemotherapy as employed herein is intended to refer to specific antineoplastic chemical agents or drugs that are “selectively” destructive to malignant cells and tissues, for example alkylating agents, antimetabolites including thymidylate synthase inhibitors, anthracyclines, anti-microtubule agents including plant alkaloids, topoisomerase inhibitors, parp inhibitors and other antitumour agents. Selectively in this context is used loosely because of course many of these agents have serious side effects.

The preferred dose may be chosen by the practitioner, based on the nature of the cancer being treated.

Examples of alkylating agents, which may be employed in the method of the present disclosure include an alkylating agent selected from nitrogen mustards, nitrosoureas, tetrazines, aziridines, platins and derivatives, and non-classical alkylating agents.

Platinum containing chemotherapeutic agent (also referred to as platins) includes, for example cisplatin, carboplatin, oxaliplatin, satraplatin, picoplatin, nedaplatin, triplatin and lipoplatin (a liposomal version of cisplatin), in particular cisplatin, carboplatin and oxaliplatin.

he dose for cisplatin ranges from about 20 to about 270 mg/m² depending on the exact cancer. Often the dose is in the range about 70 to about 100 mg/m².

Nitrogen mustards include mechlorethamine, cyclophosphamide, melphalan, chlorambucil, ifosfamide and busulfan.

Nitrosoureas include N-Nitroso-N-methylurea (MNU), carmustine (BCNU), lomustine (CCNU) and semustine (MeCCNU), fotemustine and streptozotocin. Tetrazines include dacarbazine, mitozolomide and temozolomide.

Aziridines include thiotepa, mytomycin and diaziquone (AZQ).

Examples of antimetabolites, which may be employed in the method of the present disclosure, include anti-folates (for example methotrexate and pemetrexed), purine analogues (for example thiopurines, such as azathiopurine, mercaptopurine, thiopurine, fludarabine (including the phosphate form), pentostatin and cladribine), pyrimidine analogues (for example fluoropyrimidines, such as 5-fluorouracil and prodrugs thereof such as capecitabine [Xeloda®]), floxuridine, gemcitabine, cytarabine, decitabine, raltitrexed(tomudex) hydrochloride, cladribine and 6-azauracil.

Examples of anthracyclines, which may be employed in the method of the present disclosure, include daunorubicin (Daunomycin), daunorubicin (liposomal), doxorubicin (Adriamycin), doxorubicin (liposomal), epirubicin, idarubicin, valrubicin (currently used only to treat bladder cancer) and mitoxantrone an anthracycline analog, in particular doxorubicin.

Examples of anti-microtubule agents, which may be employed in the method of the present disclosure, include include vinca alkaloids and taxanes.

Vinca alkaloids include completely natural chemicals, for example vincristine and vinblastine and also semi-synthetic vinca alkaloids, for example vinorelbine, vindesine, and vinflunine

Taxanes include paclitaxel, docetaxel, abraxane, carbazitaxel and derivatives of thereof. Derivatives of taxanes as employed herein includes reformulations of taxanes like taxol, for example in a micellar formulations, derivatives also include chemical derivatives wherein synthetic chemistry is employed to modify a starting material which is a taxane.

Topoisomerase inhibitors, which may be employed in a method of the present disclosure include type I topoisomerase inhibitors, type II topoisomerase inhibitors and type II topoisomerase poisons. Type I inhibitors include topotecan, irinotecan, indotecan and indimitecan. Type II inhibitors include genistein and ICRF 193 which has the following structure:

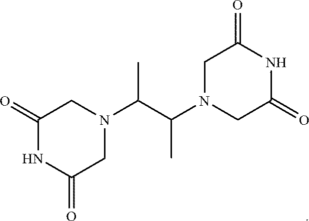

Type II poisons include amsacrine, etoposide, etoposide phosphate, teniposide and doxorubicin and fluoroquinolones.

In one embodiment a combination of chemotherapeutic agents employed is, for example a platin and 5-FU or a prodrug thereof, for example cisplatin or oxaplatin and capecitabine or gemcitabine, such as FOLFOX.

In one embodiment the chemotherapy comprises a combination of chemotherapy agents, in particular cytotoxic chemotherapeutic agents.

In one embodiment the chemotherapy combination comprises a platin, such as cisplatin and fluorouracil or capecitabine.

In one embodiment the chemotherapy combination in capecitabine and oxaliplatin (Xelox).

In one embodiment the chemotherapy is a combination of folinic acid and 5-FU, optionally in combination with oxaliplatin.

In one embodiment the chemotherapy is a combination of folinic acid, 5-FU and irinotecan (FOLFIRI), optionally in combination with oxaliplatin (FOLFIRINOX). The regimen consists of: irinotecan (180 mg/m² IV over 90 minutes) concurrently with folinic acid (400 mg/m² [or 2 × 250 mg/m²] IV over 120 minutes); followed by fluorouracil (400-500 mg/m² IV bolus) then fluorouracil (2400-3000 mg/m² intravenous infusion over 46 hours). This cycle is typically repeated every two weeks. The dosages shown above may vary from cycle to cycle.

In one embodiment the chemotherapy combination employs a microtubule inhibitor, for example vincristine sulphate, epothilone A, N-[2-[(4-Hydroxyphenyl)amino]-3-pyridinyl]-4-methoxybenzenesulfonamide (ABT-751), a taxol derived chemotherapeutic agent, for example paclitaxel, abraxane, or docetaxel or a combination thereof.

In one embodiment the chemotherapy combination comprises an antimetabolite such as capecitabine (xeloda), fludarabine phosphate, fludarabine (fludara), decitabine, raltitrexed (tomudex), gemcitabine hydrochloride and cladribine.

In one embodiment the anticancer therapy combination employs an mTor inhibitor. Examples of mTor inhibitors include: everolimus (RAD001), WYE-354, KU-0063794, papamycin (Sirolimus), Temsirolimus, Deforolimus(MK-8669), AZD8055 and BEZ235(NVP-BEZ235).

In one embodiment the anticancer therapy combination employs a MEK inhibitor. Examples of MEK inhibitors include: AS703026, CI-1040 (PD184352), AZD6244 (Selumetinib), PD318088, PD0325901, AZD8330, PD98059, U0126-EtOH, BIX 02189 or BIX 02188.

In one embodiment the chemotherapy combination employs an AKT inhibitor. Examples of AKT inhibitors include: MK-2206 and AT7867.

In one embodiment the anticancer therapy employs an aurora kinase inhibitor. Examples of aurora kinase inhibitors include: Aurora A Inhibitor I, VX-680, AZD1152-HQPA (Barasertib), SNS-314 Mesylate, PHA-680632, ZM-447439, CCT129202 and Hesperadin.

In one embodiment the chemotherapy combination employs a p38 inhibitor, for example as disclosed in WO2010/038086, such as N-[4-({4-[3-(3-tert-Butyl-1-p-tolyl-1H-pyrazol-5-yl)ureido]naphthalen-1-yloxy}methyl)pyridin-2-yl]-2-methoxyacetamide.

In one embodiment the combination employs a Bcl-2 inhibitor. Examples of Bcl-2 inhibitors include: obatoclax mesylate, ABT-737, ABT-263(navitoclax) and TW-37.

In one embodiment the chemotherapy combination comprises ganciclovir, which may assist in controlling immune responses and/or tumour vasculation.

In one embodiment the anticancer therapy includes a PARP inhibitor.

In one embodiment the anticancer therapy includes an inhibitor of cancer metabolism with specific inhibition of the activity of the DHODH enzyme.

In one embodiment one or more therapies employed in the method herein are metronomic, that is a continuous or frequent treatment with low doses of anticancer drugs, often given concomitant with other methods of therapy.

In one embodiment, there is provided the use of multiple cycles of treatment (such as chemotherapy) for example 2, 3, 4, 5, 6, 7 or 8.

In one embodiment the compound of the disclosure is not:
Pyrido(3,4-d)pyrimindin-4-amine, N-[2-(1H-benzimidazol-2-yl)ethyl]-5,6,7,8-tetrahydro-2-(4-pyridinyl) CAS registry number 1331980-89-2;
Pyrido(3,4-d)pyrimindin-4-amine, N-[2-(1H-benzimidazol-2-yl)ethyl]-5,6,7,8-tetrahydro-2-(3-pyridinyl) CAS registry number 1332109-12-2;
Pyrido(3,4-d)pyrimindin-4-amine,5,6,7,8-tetrahydro-N-[2-(1H-indol-3-yl)ethyl]-2-(3-pyridinyl) CAS registry number 1332134-49-2;
5H-Pyrrolo(3,4-d)pyrimidin-4-amine,6,7-dihydro-N-(2-(1H-indol-3-yl)ethyl)-2-(3-pyridinyl) CAS registry number 1360232-40-1;
5H-Pyrrolo(3,4-d)pyrimidin-4-amine,6,7-dihydro-N-(2-(1H-indol-1-yl)ethyl)-2-(3-pyridinyl) CAS registry number;
Pyrido(3,4-d)pyrimidin-4-amine, 5,6,7,8-tetrahydro-N-[2-(1H-indol-3-yl)ethyl]-2-phenyl CAS registry number 1360364-34-6;
Pyrido(3,4-d)pyrimidin-4-amine, 5,6,7,8-tetrahydro-N-[2-(1H-indol-3-yl)ethyl]-2-)2-pyridinyl) CAS registry number 1360407-66-4.
“Comprising” in the context of the present specification is intended to mean “including”. Where technically appropriate, embodiments of the invention may be combined.

Embodiments are described herein as comprising certain features/elements. The disclosure also extends to separate embodiments consisting or consisting essentially of said features/elements.

Technical references such as patents and applications are incorporated herein by reference.

Any embodiments specifically and explicitly recited herein may form the basis of a disclaimer either alone or in combination with one or more further embodiments.

The present application claims priority from SG10202001722X filed 26 Feb. 2020 and incorporated herein by reference. This application may be used as the basis for making corrections.

The invention will now be described with reference to the following examples, which are merely illustrative and should not be construed as limiting the scope of the present invention.

### EXAMPLES

### General Method A (Tryptamine)

A suitable round bottom flask or reacti-vial was charged with aryl halide (1 equiv.), tryptamine (1.1 equiv.), IPA (10 mL/mmol) and triethylamine (2 equiv.) and heated at 100° C. for 3 h (reaction monitored by UPLC analysis). On cooling the reaction mixture was evaporated to dryness and the resultant residue partitioned between ethyl acetate and water. The organic phase was separated and sequentially washed with saturated bicarbonate solution, water, brine, then dried over sodium sulfate, filtered and evaporated. Purification, if required was performed by chromatography or trituration.

### General Method B (Suzuki)

A suitable round bottom flask or reacti-vial was charged with aryl halide (lequiv.), aryl boronic acid (1.5-2.0 equiv.), potassium carbonate (1.5-2.0 equiv.), dioxane/water ([5:1] about 60 vol). Head space was flushed with nitrogen gas, then [1,1′-Bis(diphenylphosphino) ferrocene]dichloropalladium(II) dichloride (0.2-0.3 equiv.) was added. The reaction mixture was heated under nitrogen at 100° C. for 2-24 h until complete as determined by UPLC analysis. The reaction mixture was evaporated to dryness and applied to a silica column as a slurry in DCM; or preabsorbed onto celite, which was loaded in to a dry load unit and placed in series with a silica cartridge. The desired product was eluted with a gradient of ethyl acetate in hexane, sometimes more polar eluent of methanol (0-10%) in ethyl acetate may be required. Further chromatography on silica eluting with 7 M ammonia in methanol (0-10%) in DCM may be required. Trituration with diethyl ether and subsequent filtration afforded the desired product.

### General Method C (TFA deBOC)

TFA (0.2-0.5 mL) was added to a solution of BOC compound (20-200 mg) in DCM (3-10 mL). Once complete as judged by UPLC, the reaction mixture was loaded on to an SCX resin cartridge (0.5 g or 1.0 g). The cartridge was washed through with methanol (10 mL). The product was eluted as the free base, eluting with 7 M ammonia in methanol (10 mL). The free based material was evaporated, triturated with ether and collected by filtration. Dried in a desiccator <10 mbar.

Reference Sample 1 to 12 disclosed in the priority document SG10202001722X filed 26 Feb. 2020, are disclosed in WO2020/039093, and are incorporated herein specifically by reference.

The following Examples in Table 1 were made by methods analogous to those given herein and used for reference compounds 1 to 12:

| Eg No. | Compound | Compound |
|---|---|---|
| 1 | 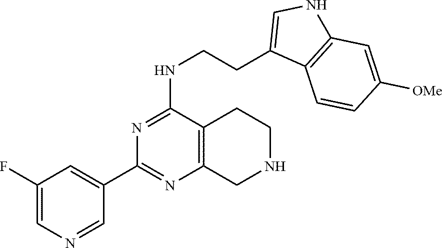 [SMILES: COc1ccc2c(CCNc3nc(-c4cncc(F)c4)nc4c3CCNC4)c[nH]c2c1] | 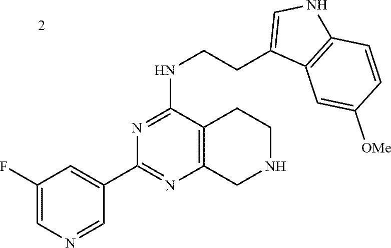 |
| 3 | 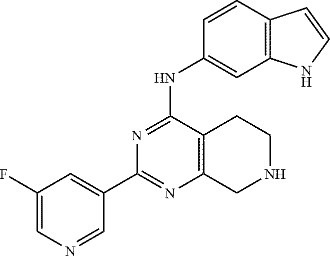 [SMILES: Fc1cncc(-c2nc3c(c(Nc4ccc5cc[nH]c5c4)n2)CCNC3)c1] | 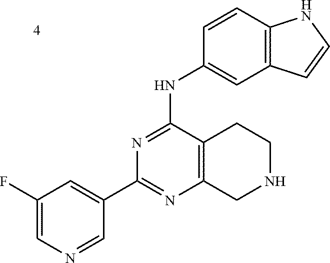 |
| 5 | 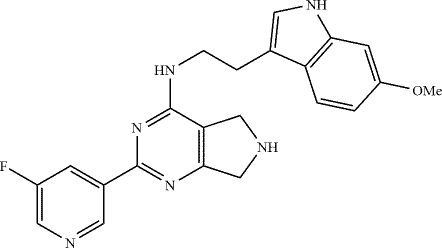 [SMILES: COc1ccc2c(CCNc3nc(-c4cncc(F)c4)nc4c3CNC4)c[nH]c2c1] | 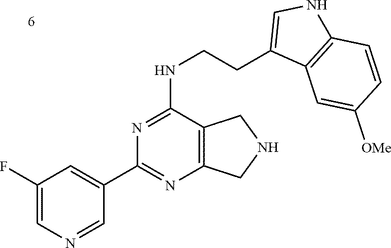 |
| 7 | 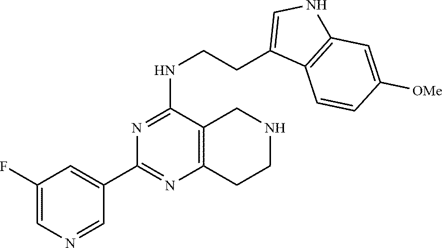 [SMILES: COc1ccc2c(CCNc3nc(-c4cncc(F)c4)nc4c3CNCC4)c[nH]c2c1] | 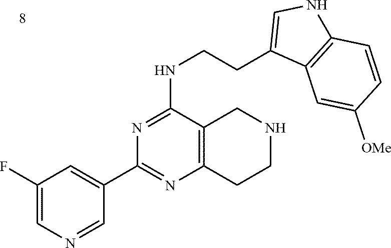 |
| 9 | 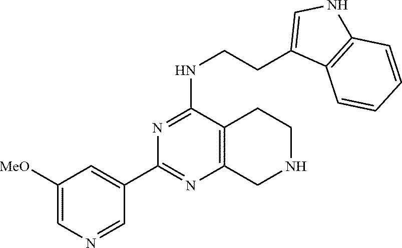 [SMILES: COc1cncc(-c2nc3c(c(NCCc4c[nH]c5ccccc45)n2)CCNC3)c1] | 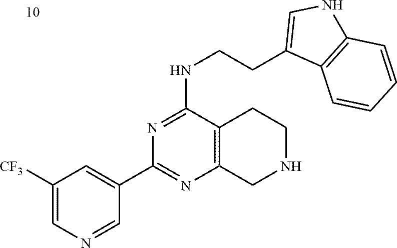 |
| 11 | 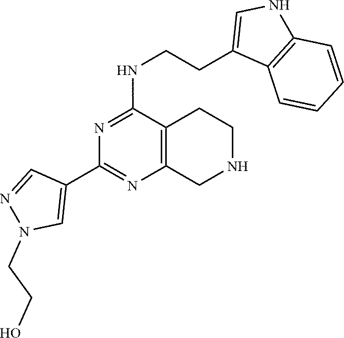 [SMILES: OCCn1cc(-c2nc3c(c(NCCc4c[nH]c5ccccc45)n2)CCNC3)cn1] | 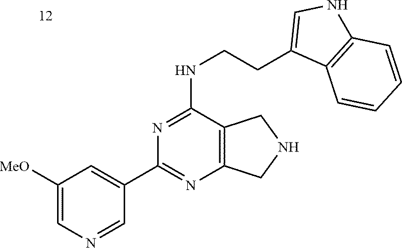 |
| 13 | 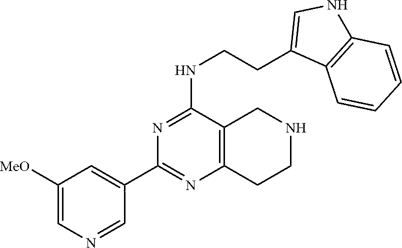 [SMILES: COc1cncc(-c2nc3c(c(NCCc4c[nH]c5ccccc45)n2)CNCC3)c1] | 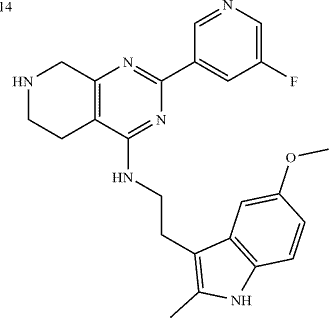 |
| 15 | 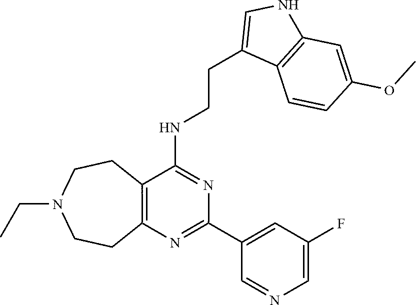 [SMILES: CCN1CCc2nc(-c3cncc(F)c3)nc(NCCc3c[nH]c4cc(OC)ccc34)c2CC1] | 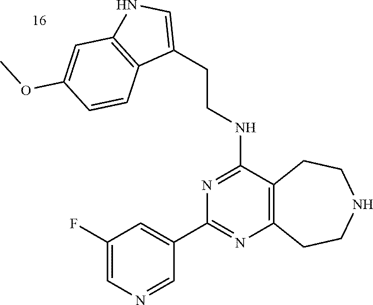 |
| 17 | 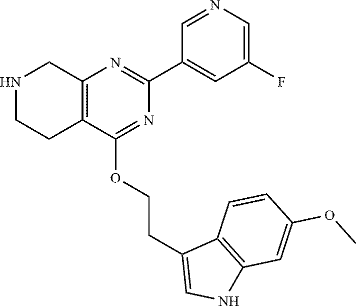 [SMILES: COc1ccc2c(CCOc3nc(-c4cncc(F)c4)nc4c3CCNC4)c[nH]c2c1] | 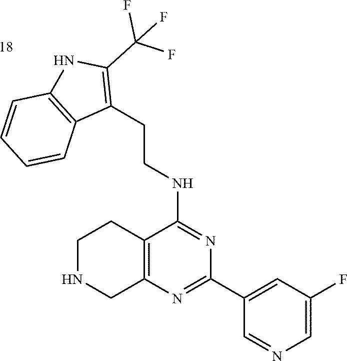 |

### Example 1

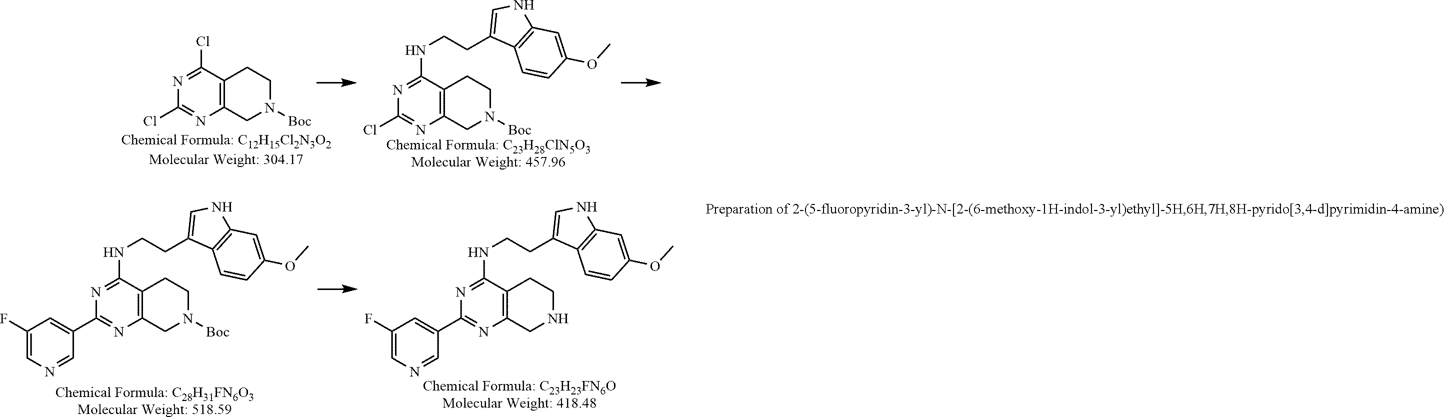

### Step 1 Tert-Butyl 2-Chloro-4-{[2-(5-Methoxy-1H-Indol-3-yl)Ethyl]Amino}-5H,6H,7H,8H-Pyrido[3,4-d]Pyrimidine-7-Carboxylate)

Prepared according to general method A, using t-butyl2,4-dichloro-5,6-dihydropyrido[3,4-d]pyrimidine-7(8H)carboxylate (500 mg) and 2-(6-methoxy-1H-indol-3-yl)ethan-1-amine (340 mg) to give the desired product, following purification by triturated from MTBE (10 mL), as a an off-white solid (510 mg, 68%)
UPLC-MS (Basic Method, 2 min): rt 1.19 min, m/z = 458/460 [M+H]+ chlorine isotope pattern
1 H NMR (400 MHz, DMSO) δ 10.74 - 10.54 (m, 1 H), 7.53 (s, 1 H), 7.23 (d, J = 8.7 Hz, 1 H), 7.14 (d, J = 2.4 Hz, 1 H), 7.05 (d, J = 2.4 Hz, 1 H), 6.72 (dd, J = 8.8, 2.4 Hz, 1 H), 4.27 (s, 2 H), 3.76 (s, 3 H), 3.67 -3.50 (m, 4 H), 2.92 (t, J = 7.6 Hz, 2 H), 2.34 (t, J = 5.7 Hz, 2 H), 1.43 (s, 9 H).

### Step 2 Tert-Butyl 2-(5-Fluoropyridin-3-yl)-4-{[2-(5-Methoxy-1H-Indol-3-yl)Ethyl]Amino}-5H,6H,7H,8H-Pyrido[3,4-d]Pyrimidine-7-Carboxylate)

Prepared according to general method B, using tert-butyl 2-chloro-4-{[2-(5-methoxy-1H-indol-3-yl)ethyl]amino}-5H,6H,7H,8H-pyrido[3,4-d]pyrimidine-7-carboxylate) (500 mg) and 5-fluoropyridine-3-boronic acid (231 mg) to give the desired product as an off-white solid (226 mg, 21%).
UPLC-MS (Basic Method, 2 min): rt 1.27 min, m/z 519.3 [M+H]+
19F NMR (400 MHz, DMSO-d6) δ ppm -127.61 proton decoupled
1 H NMR (400 MHz, DMSO) δ 10.66 (s, 1 H), 9.32 (d, J = 1.7 Hz, 1 H), 8.67 (d, J = 2.9 Hz, 1 H), 8.42 –8.19 (m, 1 H), 7.27 (s, 1 H), 7.22 (d, J = 8.7 Hz, 1 H), 7.17 (d, J = 2.3 Hz, 1 H), 7.00 (d, J = 2.4 Hz, 1 H), 6.71 (dd, J = 8.8, 2.4 Hz, 1 H), 4.40 (s, 2 H), 3.79 (q, J = 6.8 Hz, 2 H), 3.70 (s, 3 H), 3.68 – 3.57 (m, 2 H), 3.03 (t, J = 7.5 Hz, 2 H), 2.44 (t, J = 5.9 Hz, 2 H), 1.45 (s, 9 H).

### Step 3 2-(5-Fluoropyridin-3-yl)-N-[2-(6-Methoxy-1H-Indol-3-yl)Ethyl]-5H,6H,7H,8H-Pyrido[3,4-d]Pyrimidin-4-Amine)

Prepared according to general method C, using tert-butyl 2-(5-fluoropyridin-3-yl)-4-{[2-(5-methoxy-1H-indol-3-yl)ethyl]amino)-5H,6H,7H,8H-pyrido[3,4-d]pyrimidine-7-carboxylate) (300 mg) to give the desired product, following purification by preparative HPLC purification (basic conditions), as a white solid (54 mg, 23%).
UPLC-MS (4 min basic): rt = 1.57 min, m/z = 419.3 [M+H]+
¹⁹F NMR (376 MHz, DMSO) δ -127.82 proton decoupled
¹H NMR (400 MHz, DMSO) δ 10.62 (s, 1 H), 9.31 (d, J = 1.7 Hz, 1 H), 8.66 (d, J = 2.8 Hz, 1 H), 8.36 -8.24 (m, 1 H), 7.47 (d, J = 8.6 Hz, 1 H), 7.15 - 7.01 (m, 2 H), 6.85 (d, J = 2.2 Hz, 1 H), 6.64 (dd, J = 8.6, 2.3 Hz, 1 H), 3.82 – 3.73 (m, 5 H), 3.71 (s, 2 H), 2.99 (t, J = 5.1 Hz, 4 H), 2.34 (t, 2 H).

### Example 5

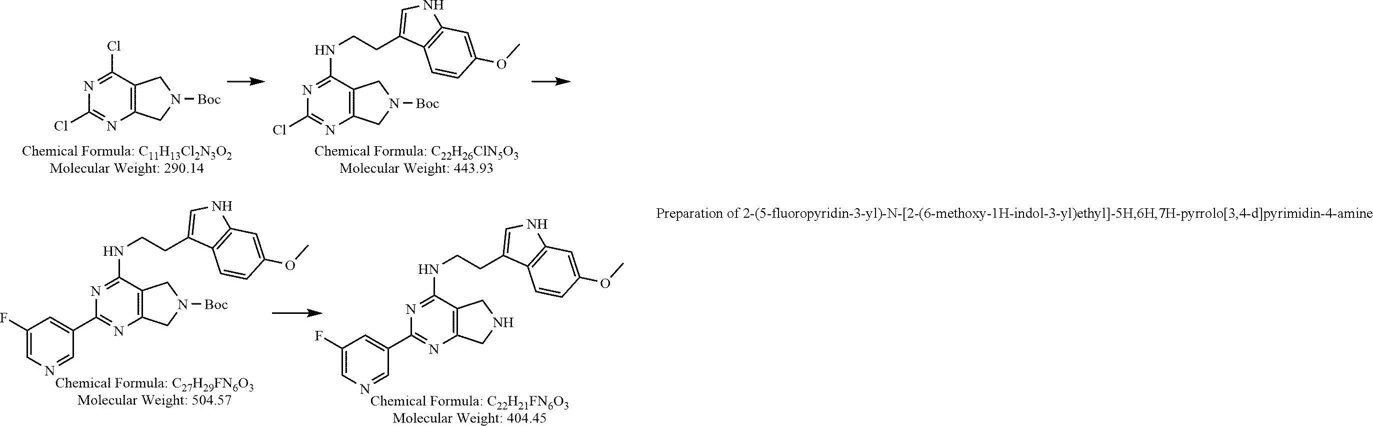

### Step 1 Tert-Butyl 2-Chloro-4-{[2-(6-Methoxy-1H-Indol-3-yl)Ethyl]Amino}-5H,6H,7H-Pyrrolo[3,4-d]Pyrimidine-6-Carboxylate

Prepared according to general method A, using tert-butyl 2,4-dichloro-5H,6H,7H-pyrrolo[3,4-d]pyrimidine-6-carboxylate (500 mg) and 2-(6-methoxy-1H-indol-3-yl)ethan-1-amine (361 mg) to give the desired product, following purification by trituration from MTBE, as a yellow solid (889 mg, 98%)
UPLC-MS (Basic Method, 2 min): rt 1.17 min, m/z 444.3/446.3 [M+H]+ chlorine isotope pattern
1 H NMR (400 MHz, DMSO) δ 10.62 (s, 1 H), 7.97 (d, J = 5.4 Hz, 1 H), 7.49 (dd, J = 8.8, 2.4 Hz, 1 H), 7.03 (d, J = 2.2 Hz, 1 H), 6.85 (d, J = 2.3 Hz, 1 H), 6.65 (dd, J = 8.6, 2.3 Hz, 1 H), 4.46 - 4.25 (m, 4 H), 3.76 (s, 3 H), 3.58 (q, J = 7.0 Hz, 2 H), 2.90 (t, J = 7.6 Hz, 2 H), 1.46 (d, J = 4.3 Hz, 9 H)

### Step 2 Tert-Butyl 2-(5-Fluoropyridin-3-yl)-4-{[2-(6-Methoxy-1H-Indol-3-yl)Ethyl]Amino}-5H,6H,7H-Pyrrolo[3,4-d]Pyrimidine-6-Carboxylate

Prepared according to general method B, using tert-butyl 2-chloro-4-{[2-(6-methoxy-1H-indol-3-yl)ethyl]amino}-5H,6H,7H-pyrrolo[3,4-d]pyrimidine-6-carboxylate (889 mg) and 5-fluoropyridine-3-boronic acid (565 mg) to give the desired product as a white solid (333 mg, 33%).
UPLC-MS (Basic Method, 2 min): rt 1.22 min, m/z 505.4 [M+H]+
19F NMR (400 MHz, DMSO-d6) δ ppm -127.58 proton decoupled
1 H NMR (400 MHz, DMSO) δ 10.61 (d, J = 2.2 Hz, 1 H), 9.31 (q, J = 1.8 Hz, 1 H), 8.69 (d, J = 2.9 Hz, 1 H), 8.31 (ddd, J = 11.7, 6.1, 3.3 Hz, 1 H), 7.71 (d, J = 4.9 Hz, 1 H), 7.45 (dd, J = 8.6, 2.2 Hz, 1 H), 7.06 (d, J = 2.2 Hz, 1 H), 6.84 (d, J = 2.2 Hz, 1 H), 6.64 (dd, J = 8.6, 2.3 Hz, 1 H), 4.45 (dd, J = 19.9, 12.6 Hz, 4 H), 3.75 (s, 5 H), 2.98 (t, J = 7.4 Hz, 2 H), 1.48 (d, J = 4.4 Hz, 9 H)

### Step 3 2-(5-Fluoropyridin-3-yl)-N-[2-(6-<Ethoxy-1H-Indol-3-yl)Ethyl]-5H,6H,7H-Pyrrolo[3,4-d]Pyrimidin-4-Amine

Prepared according to general method C, using tert-butyl 2-(5-fluoropyridin-3-yl)-4-{[2-(6-methoxy-1H-indol-3-yl)ethyl]amino}-5H,6H,7H-pyrrolo[3,4-d]pyrimidine-6-carboxylate (333 mg) to give the desired product, following triturated from MTBE, as a white solid (120 mg, 45%).
UPLC-MS (4 min basic): rt = 1.40 min, m/z = 405.3 [M+H]+
19F NMR (376 MHz, DMSO) δ -127.73 (s)
1 H NMR (400 MHz, DMSO) δ 10.66 (d, J = 2.3 Hz, 1 H), 9.37 (t, J = 1.7 Hz, 1 H), 8.71 (d, J = 2.9 Hz, 1 H), 8.38 (ddd, J = 10.1, 2.9, 1.6 Hz, 1 H), 7.50 (d, J = 8.6 Hz, 1 H), 7.46 (t, J = 5.8 Hz, 1 H), 7.10 (d, J = 2.2 Hz, 1 H), 6.89 (d, J = 2.3 Hz, 1 H), 6.68 (dd, J = 8.6, 2.3 Hz, 1 H), 4.03 (dd, J = 7.0, 2.1 Hz, 4 H), 3.80 (s, 5 H), 3.03 (dd, J = 8.6, 6.4 Hz, 2 H)

### Example 9

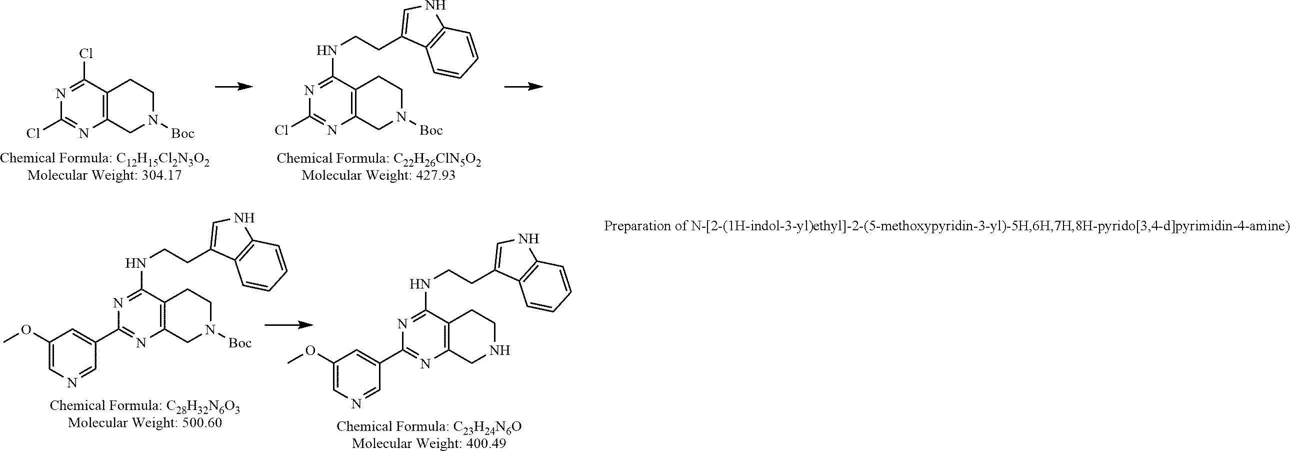

### Step 1 Tert-Butyl 2-Chloro-4-{[2-(1H-Indol-3-yl)Ethyl]Amino}-5H,6H,7H,8H-Pyrido[3,4-d]Pyrimidine-7-Carboxylate)

Prepared according to general method A, using tert-butyl 2,4-dichloro-5,6-dihydropyrido[3,4-d]pyrimidine-7(8H)carboxylate (500 mg) and tryptamine (333 mg) to give the desired product, following purification by triturated from MTBE (10 mL), as a white solid (345 mg, 49%)
UPLC-MS (Basic Method, 2 min): rt 1.23 min, m/z 428.3/430.3 [M+H]+ chlorine isotope pattern
H NMR (400 MHz, DMSO) δ 10.81 (s, 1 H), 7.64 (d, J = 7.8 Hz, 1 H), 7.53 (s, 1 H), 7.34 (d, J = 8.0 Hz, 1 H), 7.18 (d, J = 2.3 Hz, 1 H), 7.06 (td, J = 8.1, 7.0, 1.2 Hz, 1 H), 6.98 (td, J = 7.4, 6.9, 1.0 Hz, 1 H), 4.27 (s, 2 H), 3.59 (p, J = 5.9, 5.3 Hz, 4 H), 2.95 (t, J = 7.6 Hz, 2 H), 2.35 (t, J = 5.9 Hz, 2 H), 1.42 (s, 9 H).

### Step 2 Tert-Butyl 4-{[2-(1H-indol-3-yl)Ethyl]Amino}-2-(5-Methoxypyridin-3-yl)-5H,6H,7H,8H-Pyrido[3,4-d]Pyrimidine-7-Carboxylate)

Prepared according to general method B, using 1 tert-butyl 2-chloro-4-{[2-(1H-indol-3-yl)ethyl]amino}-5H,6H,7H,8H-pyrido[3,4-d]pyrimidine-7-carboxylate) (100 mg) and (5-methoxypyridin-3-yl)boronic acid) (54 mg) to give the desired product as a white solid (58 mg, 25%).
UPLC-MS (Basic Method, 2 min): rt 1.21 min, m/z 501.4 [M+H]+
1 H NMR (400 MHz, DMSO) δ 10.84 (s, 1 H), 9.09 (d, J = 1.6 Hz, 1 H), 8.38 (d, J = 2.9 Hz, 1 H), 8.11 (dd, J = 3.0, 1.7 Hz, 1 H), 7.59 (d, J = 7.8 Hz, 1 H), 7.35 (d, J = 8.1 Hz, 1 H), 7.26 (s, 1 H), 7.22 (d, J = 2.1 Hz, 1 H), 7.07 (t, J = 7.4 Hz, 1 H), 6.96 (t, J = 7.5 Hz, 1 H), 4.41 (s, 2 H), 3.87 (s, 3 H), 3.80 (q, J = 7.0 Hz, 2 H), 3.65 (s, 2 H), 3.05 (t, J = 7.6 Hz, 2 H), 2.45 (d, J = 5.8 Hz, 2 H), 1.45 (s, 9 H).

### Step 3 N-[2-(1H-Indol-3-yl)Ethyl]-2-(5-Methoxypyridin-3-yl)-5H,6H,7H,8H-Pyrido [3,4-d]Pyrimidin-4-Amine)

Prepared according to general method C, using tert-butyl 4-{[2-(1H-indol-3-yl)ethyl]amino}-2-(5-methoxypyridin-3-yl)-5H,6H,7H,8H-pyrido[3,4-d]pyrimidine-7-carboxylate) (43 mg) to give the desired product as a beige solid (23 mg, 50%).
UPLC-MS (4 min basic): rt = 1.52 min, m/z = 401.3 [M+H]+
1 H NMR (400 MHz, DMSO-D6) δ 10.82 (s, 1 H), 9.08 (d, J = 1.6 Hz, 1 H), 8.36 (d, J = 2.9 Hz, 1 H), 8.10 (dd, J = 3.0, 1.6 Hz, 1 H), 7.60 (d, J = 7.8 Hz, 1 H), 7.38 - 7.31 (m, 1 H), 7.21 (d, J = 2.2 Hz, 1 H), 7.11 –7.03 (m, 2 H), 6.97 (ddd, J = 8.0, 7.0, 1.0 Hz, 1 H), 3.87 (s, 3 H), 3.79 (d, J = 10.9 Hz, 4 H), 3.05 (d, J = 7.1 Hz, 4 H), 2.37 (s, 2 H). One exchangeable proton missing

### Example 10

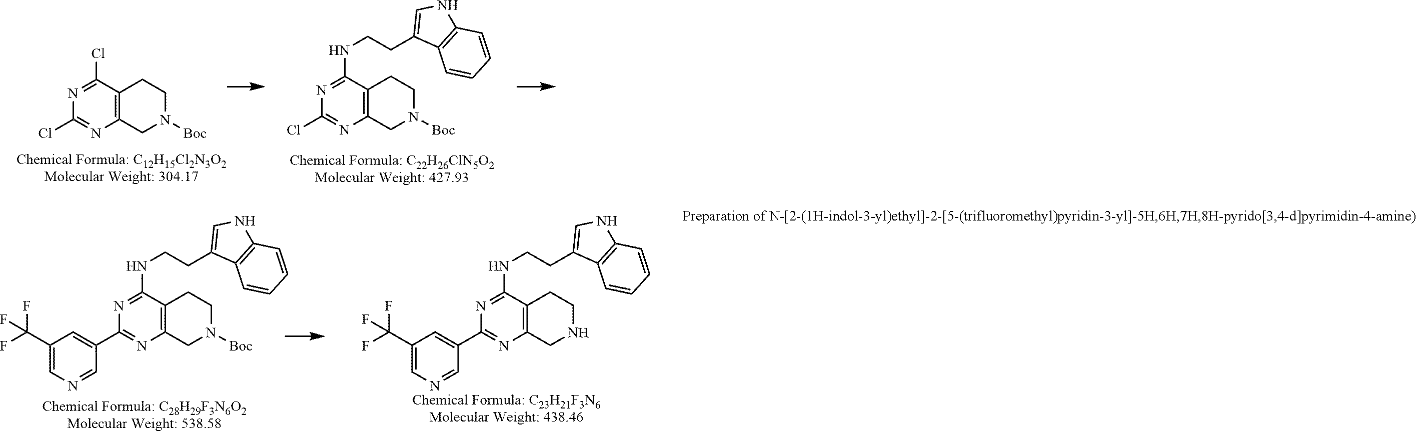

### Step 1 Tert-Butyl 2-Chloro-4-{[2-(1H-Indol-3-yl)Ethyl]Amino}-5H,6H,7H,8H-Pyrido[3,4-d]Pyrimidine-7-Carboxylate)

Prepared according to general method A, using t-butyl2,4-dichloro-5,6-dihydropyrido[3,4-d]pyrimidine-7(8H)carboxylate (500 mg) and tryptamine (333 mg) to give the desired product, following purification by triturated from MTBE (10 mL), as a white solid (345 mg, 49%)
UPLC-MS (Basic Method, 2 min): rt 1.23 min, m/z 428.3/430.3 [M+H]+ chlorine isotope pattern
H NMR (400 MHz, DMSO) δ 10.81 (s, 1 H), 7.64 (d, J = 7.8 Hz, 1 H), 7.53 (s, 1 H), 7.34 (d, J = 8.0 Hz, 1 H), 7.18 (d, J = 2.3 Hz, 1 H), 7.06 (td, J = 8.1, 7.0, 1.2 Hz, 1 H), 6.98 (td, J = 7.4, 6.9, 1.0 Hz, 1 H), 4.27 (s, 2 H), 3.59 (p, J = 5.9, 5.3 Hz, 4 H), 2.95 (t, J = 7.6 Hz, 2 H), 2.35 (t, J = 5.9 Hz, 2 H), 1.42 (s, 9 H).

### Step 2 Tert-Butyl 4-{[2-(1H-Indol-3-yl)Ethyl]amino}-2-[5-(Trifluoromethyl)Pyridin-3-yl]-5H,6H,7H,8H-Pyrido[3,4-d]Pyrimidine-7-Carboxylate)

Prepared according to general method B, using tert-butyl 2-chloro-4-{[2-(1H-indol-3-yl)ethyl]amino}-5H,6H,7H,8H-pyrido[3,4-d]pyrimidine-7-carboxylate) (100 mg) and 3-(4,4,5,5-tetramethyl-1,3,2-dioxaborolan-2-yl)-5-(trifluoromethyl)pyridine) (96 mg) to give the desired product as a white solid (53 mg, 42%).
UPLC-MS (Basic Method, 2 min): rt 1.35 min, m/z 539.3 [M+H]+
19F NMR (400 MHz, DMSO-d6) δ ppm -60.94 proton decoupled
1 H NMR (400 MHz, DMSO) δ 10.84 (s, 1 H), 9.71 (d, J = 1.9 Hz, 1 H), 9.09 (d, J = 2.2 Hz, 1 H), 8.84 (s, 1 H), 7.58 (d, J = 8.0 Hz, 1 H), 7.35 (s, 1 H), 7.35 – 7.32 (m, 1 H), 7.21 (d, J = 2.2 Hz, 1 H), 7.10 – 7.03 (m, 1 H), 7.00 – 6.93 (m, 1 H), 4.43 (s, 2 H), 3.79 (d, J = 9.6 Hz, 2 H), 3.65 (s, 2 H), 3.05 (t, J = 7.7 Hz, 2 H), 2.46 (s, 2 H), 1.45 (s, 9 H).

### Step 3 N-[2-(1H-Indol-3-yl)Ethyl]-2-[5-(Trifluoromethyl)Pyridin-3-yl]-5H,6H,7H,8H-Pyrido[3,4-d]Pyrimidin-4-Amine)

Prepared according to general method C, using tert-butyl 4-{[2-(1H-indol-3-yl)ethyl]amino}-2-[5-(trifluoromethyl)pyridin-3-yl]-5H,6H, 7H,8H -pyrido[3,4-d]pyrimidine-7 -carboxylate) (52 mg) to give the desired product as a residue (26 mg, 61%).
UPLC-MS (4 min basic): rt = 1.82 min, m/z = 439.3 [M+H]+
¹⁹F NMR (376 MHz, DMSO) δ -60.97 proton decoupled
¹H NMR (400 MHz, DMSO) δ 10.84 (s, 1 H), 9.70 (d, J = 1.9 Hz, 1 H), 9.07 (dd, J = 2.3, 1.0 Hz, 1 H), 8.83 (q, J = 3.2, 2.3 Hz, 1 H), 7.60 (d, J = 7.8 Hz, 1 H), 7.38 - 7.29 (m, 2 H), 7.20 (d, J = 2.2 Hz, 1 H), 7.07 (td, J = 8.0, 7.0, 1.1 Hz, 1 H), 6.97 (td, J = 8.0, 7.0, 1.1 Hz, 1 H), 3.90 (s, 2 H), 3.80 (q, J = 7.4, 7.0 Hz, 2 H), 3.17 (s, 2 H), 3.05 (t, J = 7.7 Hz, 2 H), 2.47 (s, 2 H). (1 H missing, NH)

### Example 14

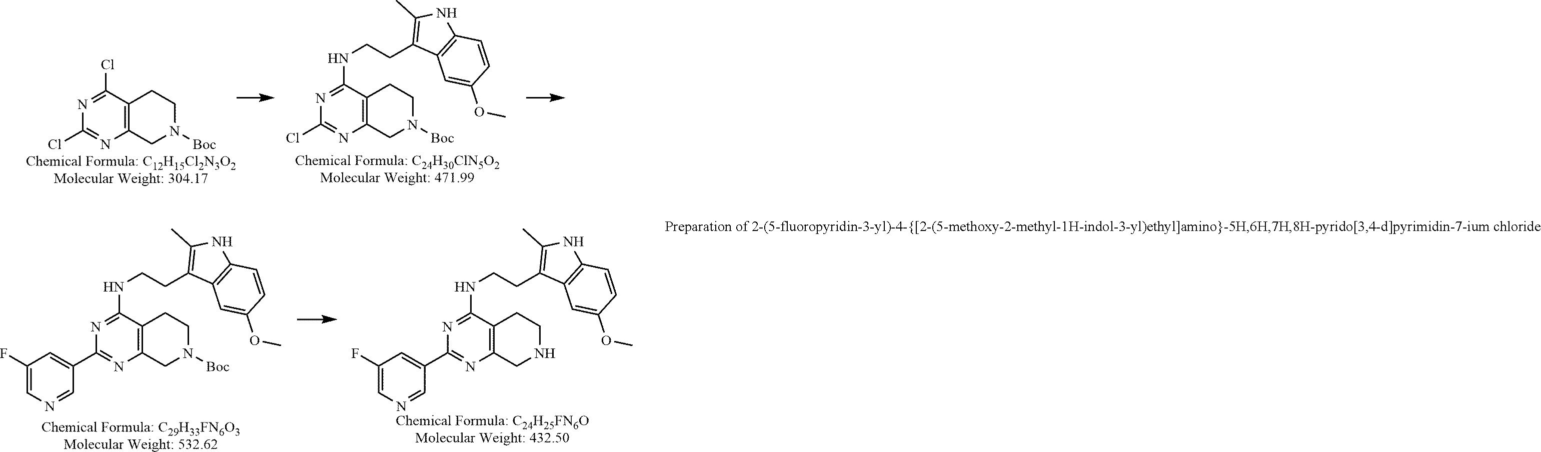

### Step 1 Tert-butyl 2-Chloro-4-{[2-(5-MMethoxy-2-Methyl-1H-Indol-3-yl)Ethyl]Amino}-5H,6H,7H,8H-Pyrido[3,4-d]Pyrimidine-7-Carboxylate

Prepared according to general method A, using tert-butyl 2,4-dichloro-5,6-dihydropyrido[3,4-d]pyrimidine-7(8H)carboxylate (500 mg) and 2-(5-methoxy-2-methyl-1H-indol-3-yl)ethan-1-amine (401 mg) to give the desired product after purification using Biotage (Telos column 10 g, Eluent Hexane-EtOAc 5 to 30 to 50%) as a brown solid (325 mg, 42%)

UPLC-MS (Basic Method, 4 min): rt 2.02 min, m/z 472.3/474.3 [M+H]+ chlorine isotope pattern 1 H NMR (400 MHz, DMSO) δ 10.58 (s, 1 H), 7.56 (s, 1 H), 7.10 (d, J = 8.6 Hz, 1 H), 6.93 (d, J = 2.4 Hz, 1 H), 6.60 (dd, J = 8.7, 2.4 Hz, 1 H), 4.26 (s, 2 H), 3.72 (s, 3 H), 3.55 (s, 2 H), 3.45 (q, J = 6.9 Hz, 2 H), 2.84 (t, J = 7.6 Hz, 2 H), 2.30 (s, 5 H), 1.42 (s, 9 H).

### Step 2 Tert-Butyl 2-(5-Fluoropyridin-3-yl)-4-{[2-(5-Methoxy-2-Methyl-1H-Indol-3-yl)Ethyl]Amino}-5H,6H,7H,8H-Pyrido[3,4-d]Pyrimidine-7-Carboxylate

Prepared according to general method B, using tert-butyl 2-chloro-4-{[2-(5-methoxy-2-methyl-1H-indol-3-yl)ethyl]amino)-5H,6H,7H,8H-pyrido[3,4-d]pyrimidine-7-carboxylate (325 mg) and 5-fluoropyridine-3-boronic acid (97 mg) to give the desired product as a brown solid (210 mg, 55%). UPLC-MS (Basic Method, 4 min): rt 1.73 min, m/z 533.3 [M+H]+
19F NMR (376 MHz, DMSO) δ -127.73. Proton decoupled
1 H NMR (400 MHz, DMSO) δ 10.53 (s, 1 H), 9.30 (d, J = 1.8 Hz, 1 H), 8.65 (d, J = 2.8 Hz, 1 H), 8.26 (dt, J = 10.1, 2.4 Hz, 1 H), 7.26 (s, 1 H), 7.09 (d, J = 8.6 Hz, 1 H), 6.90 (d, J = 2.4 Hz, 1 H), 6.60 (dd, J = 8.6, 2.4 Hz, 1 H), 4.38 (s, 2 H), 3.68 (s, 3 H), 3.67 – 3.56 (m, 4 H), 2.93 (t, J = 7.4 Hz, 2 H), 2.40 (t, J = 5.9 Hz, 2 H), 2.30 (s, 3 H), 1.44 (s, 9 H).

### Step 3 2-(5-Fluoropyridin-3-yl)-4-{[2-(5-Methoxy-2-Methyl-1H-Indol-3-yl)Ethyl]Amino}-5H,6H,7H,8H-Pyrido[3,4-d]Pyrimidin-7-ium Chloride

tert-Butyl 2-(5-fluoropyridin-3-yl)-4-{[2-(5-methoxy-2-methyl-1H-indol-3-yl)ethyl]amino}-5H,6H,7H,8H-pyrido[3,4-d]pyrimidine-7-carboxylate (210 mg, 0.39 mmol, 1.0 eq) was treated with 4 M HCl in dioxane (4.0 mL, 16.0 mmol, 40.6 eq), and the resulting mixture was stirred for 3 h. The resulting suspension was filtered and the solid was washed with ether before being dried under suction to give the desire product as a solid (175 mg, 91%)
UPLC-MS (4 min Basic): rt = 1.58 min, m/z = 433.2 [M+H]+
19F NMR (376 MHz, DMSO) δ -126.81 (d, J = 9.7 Hz) proton decoupled
1 H NMR (400 MHz, DMSO) δ 10.57 (s, 1 H), 9.91 (s, 2 H), 9.29 (d, J = 1.7 Hz, 1 H), 8.75 (d, J = 2.8 Hz, 1 H), 8.37 – 8.28 (m, 1 H), 8.02 (s, 1 H), 7.08 (d, J = 8.7 Hz, 1 H), 6.96 (d, J = 2.4 Hz, 1 H), 6.60 (dd, J = 8.7, 2.4 Hz, 1 H), 4.20 (t, J = 4.4 Hz, 2 H), 3.71 (s, 5 H), 3.43 (d, J = 6.5 Hz, 2 H), 2.95 (t, J = 7.5 Hz, 2 H), 2.70 (t, J = 6.1 Hz, 2 H), 2.30 (s, 3 H)

### Example 15 & 16

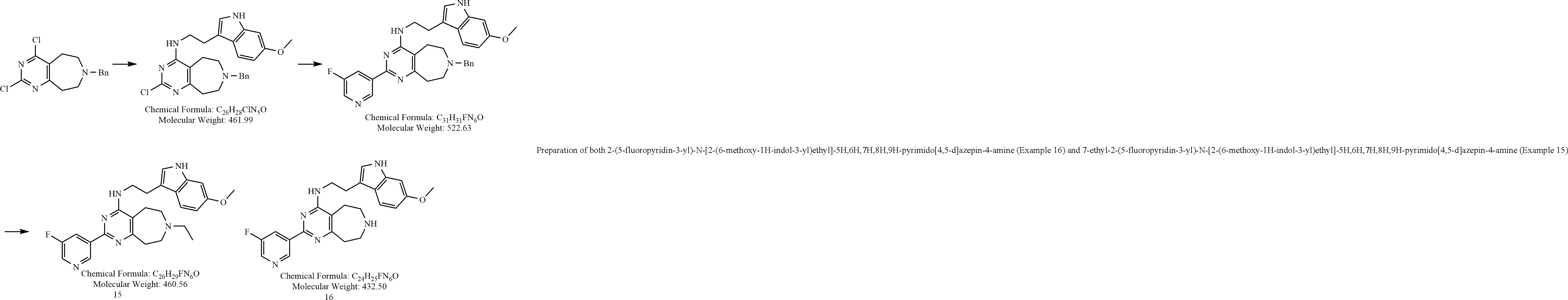

### Step 1 7-Benzyl-2-Chloro-N-[2-(6-Methoxy-1H-Indol-3-yl)Ethyl]-5H,6H,7H,8H,9H-Pyrimido[4,5-d]Azepin-4-Amine

Prepared according to general method A, using benzyl 2,4-dichloro-5,6,8,9-tetrahydro-7H-pyrimido[4,5-d]azepine-7-carboxylate (131 mg) and 2-(6-methoxy-1H-indol-3-yl)ethan-1-amine (80 mg) to give the desired product, following purification by automated column chromatography using Biotage (Telos column 25 g, eluting with a gradient of EtOAc in heptane, 60 to 80% EtOAc) as a white solid (96 mg, 44%)
UPLC-MS (Basic, 2 min): rt 1.22 min, m/z 462.4/464.4 [M+H]+ chlorine isotope pattern.
1 H NMR (400 MHz, DMSO) δ 10.60 (s, 1 H), 7.50 (d, J = 8.6 Hz, 1 H), 7.44 (t, J = 5.5 Hz, 1 H), 7.34 (d, J = 4.4 Hz, 4 H), 7.29 - 7.24 (m, 1 H), 7.01 (d, J = 2.2 Hz, 1 H), 6.84 (d, J = 2.3 Hz, 1 H), 6.64 (dd, J = 8.6, 2.3 Hz, 1 H), 3.76 (s, 3 H), 3.61 (s, 2 H), 3.53 (q, J = 6.2, 5.5 Hz, 2 H), 2.88 (t, J = 7.6 Hz, 2 H), 2.84 - 2.79 (m, 2 H), 2.70 – 2.63 (m, 2 H), 2.55 (d, J = 9.2 Hz, 2 H). Missing one CH2 (likely to be under DMSO peak).

### Step 2 7-Benzyl-2-(5-Fluoropyridin-3-yl)-N-[2-(6-Methoxy-1H-Indol-3-yl)Ethyl]-5H,6H,7H,8H,9H-Pyrimido[4,5-d]Azepin-4-Amine

Prepared according to general method B, using 7-benzyl-2-chloro-N-[2-(6-methoxy-1H-indol-3-yl)ethyl]-5H,6H,7H,8H,9H-pyrimido[4,5-d]azepin-4-amine (96 mg) and (5-fluoropyridin-3-yl)boronic acid (37 mg, 1.50 eq) to give the desired product, following trituration from MTBE, as a white solid (51 mg, 56%).
UPLC-MS (Basic Method, 2 min): rt 1.31 min, m/z 523.4 [M+H]+
19F NMR (376 MHz, DMSO) δ -127.79 proton decoupled
1 H NMR (400 MHz, DMSO) δ 10.62 (s, 1 H), 9.32 (t, J = 1.7 Hz, 1 H), 8.65 (d, J = 2.9 Hz, 1 H), 8.32 (ddd, J = 10.1, 2.9,1.6 Hz, 1 H), 7.43 (d, J = 8.6 Hz, 1 H), 7.37 – 7.32 (m, 4 H), 7.30 – 7.22 (m, 2 H), 7.04 (d, J = 2.2 Hz, 1 H), 6.84 (d, J = 2.2 Hz, 1 H), 6.62 (dd, J = 8.6, 2.3 Hz, 1 H), 3.76 (s, 4 H), 3.64 (s, 2 H), 2.98 (t, J = 7.3 Hz, 4 H), 2.75 (s, 2 H), 2.64 – 2.58 (m, 2 H)

### Step 3 2-(5-Fluoropyridin-3-yl)-N-[2-(6-Methoxy-1H-Indol-3-yl)Ethyl]-5H,6H,7H,8H,9H-Pyrimido[4,5-d]Azepin-4-Amine and 7-Ethyl-2-(5-Fluoropyridin-3-yl)-N-[2-(6-Methoxy-1H-Indol-3-yl)Ethyl]-5H,6H,7H,8H,9H-Pyrimido[4,5-d]Azepin-4-Amine

A slurry of 10% palladium on carbon (20 mg) in ethanol (1 mL) was added, under nitrogen to a solution of 7-benzyl-2-(5-fluoropyridin-3-yl)-N-[2-(6-methoxy-1H-indol-3-yl)ethyl]-5H,6H,7H,8H,9H-pyrimido[4,5-d]azepin-4-amine (51 mg) in THF (1 mL), and the resulting mixture was hydrogenated at 1 atm for 24 h. The catalyst was filtered off through a celite plug and washed through with further ethanol. The filtrate was evaporated to a minimal volume and purified by preparative HPLC (basic eluent) to afford the desired products 2-(5-fluoropyridin-3-yl)-N-[2-(6-methoxy-1H-indol-3-yl)ethyl]-5H,6H,7H,8H,9H-pyrimido[4,5-d]azepin-4-amine (9.4 mg, 22.1%) and 7-ethyl-2-(5-fluoropyridin-3-yl)-N-[2-(6-methoxy-1H-indol-3-yl)ethyl]-5H,6H,7H,8H,9H-pyrimido[4,5-d]azepin-4-amine (2.8 mg, 6.2%) as white solids

### Data for 2-(5-Fluoropyridin-3-yl)-N-[2-(6-Methoxy-1H-Indol-3-yl)Ethyl]-5H,6H,7H,8H,9H-Pyrimido[4,5-d]Azepin-4-Amine (Example 16)

UPLC-MS (4 min basic): rt = 1.58 min, m/z = 433.5 M+H]+
19F NMR (376 MHz, DMSO) δ -127.79 Proton decoupled
1 H NMR (400 MHz, DMSO) δ 10.57 (s, 1 H), 9.25 (t, J = 1.7 Hz, 1 H), 8.59 (d, J = 2.9 Hz, 1 H), 8.25 (ddd, J = 10.1,2.9,1.6 Hz, 1 H), 7.37 (d,J = 8.6 Hz, 1 H), 7.17 (t,J = 5.6 Hz, 1 H), 6.98 (d,J = 2.2 Hz, 1 H),
6.77 (d, J = 2.2 Hz, 1 H), 6.55 (dd, J = 8.6, 2.3 Hz, 1 H), 3.68 (m, 4 H), 2.89 (dd, J = 17.1, 8.7 Hz, 3 H), 2.78 – 2.70 (m, 2 H), 2.70 – 2.62 (m, 3 H), 2.51 (m, 3 H) (One exchangeable proton not seen).

### Data for 7-Ethyl-2-(5-Fluoropyridin-3-yl)-N-[2-(6-Methoxy-1H-Indol-3-yl)Ethyl] 5H,6H,7H,8H,9H-Pyrimido[4,5-d]Azepin-4-Amine (Example 15)

UPLC-MS (4 min basic): rt = 1.86 min, m/z = 461.4 [M+H]+
19F NMR (376 MHz, DMSO) δ -127.76 Proton decoupled
1 H NMR (400 MHz, DMSO) δ 10.65 (s, 1 H), 9.33 (t, J = 1.7 Hz, 1 H), 8.66 (d, J = 2.9 Hz, 1 H), 8.33 (ddd, J =10.1, 2.9, 1.6 Hz, 1 H), 7.43 (d, J = 8.6 Hz, 1 H), 7.30 (t, J = 5.7 Hz, 1 H), 7.05 (d, J = 2.2 Hz, 1 H), 6.84 (d, J = 2.2 Hz, 1 H), 6.62 (dd, J = 8.6, 2.3 Hz, 1 H), 3.78 – 3.68 (m, 4 H), 3.03 - 2.91 (m, 4 H), 2.73 (s, 2 H), 2.65 – 2.57 (m, 2 H), 1.03 (t, J = 7.1 Hz, 3 H)

### Example 17

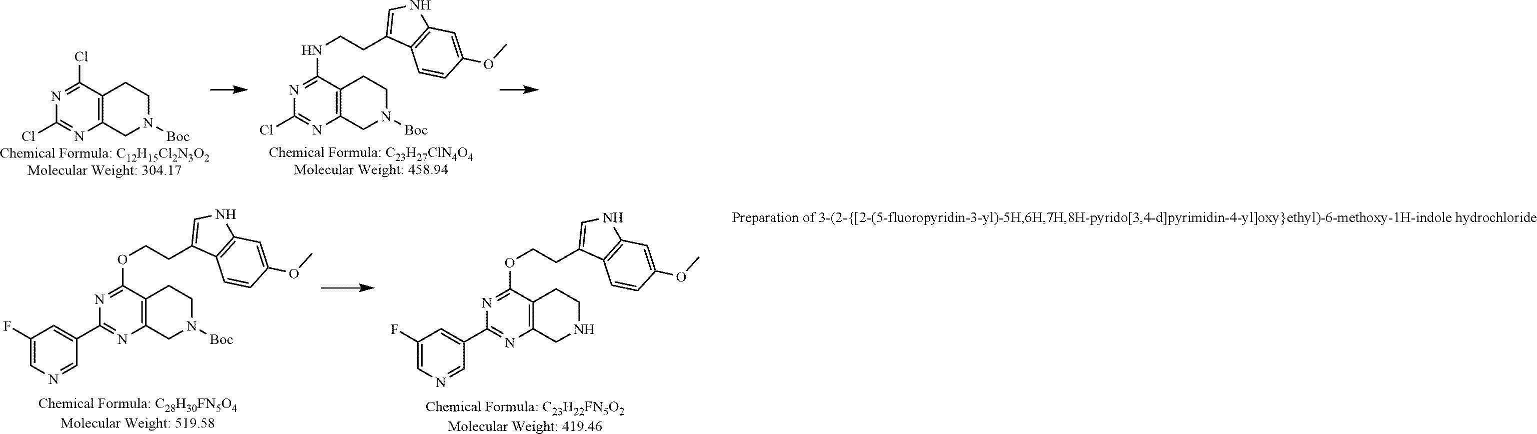

### Step 1 Tert-Butyl 2-Chloro-4-[2-(6-Methoxy-1H-Indol-3-yl)Ethoxy]-5H,6H,7H,8H-Pyrido[3,4-d]Pyrimidine-7-Carboxylate

A stirred solution of 2-(6-methoxy-1H-indol-3-yl)ethan-1-ol (321 mg, 1.01 eq) in THF (10 mL) was treated with sodium hydride (86 mg, 1.30 eq, 60% dispersion in mineral oil) at ambient temperature, and the resulting mixture was stirred for 10 min. A solution of tert-butyl 2,4-dichloro-5H,6H,7H,8H-pyrido[3,4-d]pyrimidine-7-carboxylate (505 mg, 1.0 eq) in THF (10 mL) was then added to the reaction vessel in a dropwise manner over 5 min, and the resulting mixture was stirred for 2 h at ambient temperature. The reaction was cooled down and evaporated to dryness to give a residue, which was dissolved in EtOAc and washed with water. The organic phase was evaporated to dryness to give a residue, which purified by column chromatography (Telos 120 g cartridge), eluting with a gradient of EtOAc in Heptane (10 to 30% EtOAc) to afford the desired product as a white solid (109 mg, 14%).

UPLC-MS (Basic Method, 2 min): rt 1.26 min, m/z 459.4/461.4 [M+H]+ chlorine isotope pattern 1 H NMR (400 MHz, DMSO) δ 10.71 - 10.63 (m, 1 H), 7.47 (d, J = 8.6 Hz, 1 H), 7.09 (d, J = 2.3 Hz, 1 H), 6.85 (d, J = 2.2 Hz, 1 H), 6.66 (dd, J = 8.6, 2.3 Hz, 1 H), 4.56 (t, J = 6.9 Hz, 2 H), 4.42 (s, 2 H), 3.76 (s, 3 H), 3.57 (t, J = 5.8 Hz, 2 H), 3.30 (s, 2 H), 3.11 (t, J = 6.9 Hz, 2 H), 1.43 (s, 9 H)

### Step 2 Tert-Butyl 2-(5-Fluoropyridin-3-yl)-4-[2-(6-Methoxy-1H-Indol-3-yl)Ethoxy]-5H,6H,7H,8H-Pyrido[3,4-d]Pyrimidine-7-Carboxylate

Prepared according to general method B, using tert-butyl 2-chloro-4-[2-(6-methoxy-1H-indol-3-yl)ethoxy]-5H,6H,7H,8H-pyrido[3,4-d]pyrimidine-7-carboxylate (109 mg) and 5-fluoropyridine-3-boronic acid (44 mg) to give the desired product as a white solid (81 mg, 59%).
UPLC-MS (Basic Method, 2 min): rt 1.32 min, m/z 519.6 [M+H]+
19F NMR (400 MHz, DMSO-d6) δ ppm -127.24 proton decoupled
1 H NMR (400 MHz, DMSO) δ 10.66 (d, J = 2.4 Hz, 1 H), 9.31 (t, J = 1.7 Hz, 1 H), 8.72 (d, J = 2.8 Hz, 1 H), 8.36 (ddd, J = 9.9, 2.9,1.6 Hz, 1 H), 7.46 (d, J = 8.6 Hz, 1 H), 7.11 (d, J = 2.3 Hz, 1 H), 6.84 (d, J = 2.3 Hz, 1 H), 6.64 (dd, J = 8.6, 2.3 Hz, 1 H), 4.75 (t, J = 6.9 Hz, 2 H), 4.53 (s, 2 H), 3.75 (s, 3 H), 3.70 - 3.58 (m, 2 H), 3.17 (t, J = 6.8 Hz, 2 H), 2.61 (t, J = 5.9 Hz, 2 H), 1.45 (s, 9 H)

### Step 3 3-(2-{[2-(5-Fluoropyridin-3-yl)-5H,6H,7H,8H-Pyrido[3,4-d]Pyrimidin-4-yl]Oxy}Ethyl)-6-Methoxy-1H-Indole Hydrochloride

A stirred solution of tert-butyl 2-(5-fluoropyridin-3-yl)-4-[2-(6-methoxy-1H-indol-3-yl)ethoxy]-5H,6H,7H,8H-pyrido[3,4-d]pyrimidine-7-carboxylate (81 mg) in DCM (1 mL) was treated with a 4N solution of hydrogen chloride in dioxane (0.391 mL, 10 eq), and the resulting mixture was stirred for 2 h. Thre reaction was evaporated to dryness to give a solid, which triturated with MTBE to afford the desired product as an off-white solid (76 mg, 100%).
UPLC-MS (4 min basic): rt = 1.98 min, m/z = 420.3 [M+H]+
19F NMR (376 MHz, DMSO) δ -126.97 proton decoupled
1 H NMR (400 MHz, DMSO) δ 10.72 (d, J = 2.3 Hz, 1 H), 9.67 (s, 2 H), 9.33 (t, J = 1.6 Hz, 1 H), 8.77 (d, J = 2.8 Hz, 1 H), 8.39 (ddd, J = 9.9, 2.9,1.6 Hz, 1 H), 7.48 (d, J = 8.7 Hz, 1 H), 7.12 (d, J = 2.2 Hz, 1 H), 6.84 (d, J = 2.2 Hz, 1 H), 6.66 (dd, J = 8.6, 2.3 Hz, 1 H), 4.79 (t, J = 6.8 Hz, 2 H), 4.34 – 4.26 (m, 2 H), 3.75 (s, 3 H), 3.47 – 3.40 (m, 2 H), 3.19 (t, J = 6.7 Hz, 2 H), 2.83 (t, J = 6.2 Hz, 2 H)

### Example 18

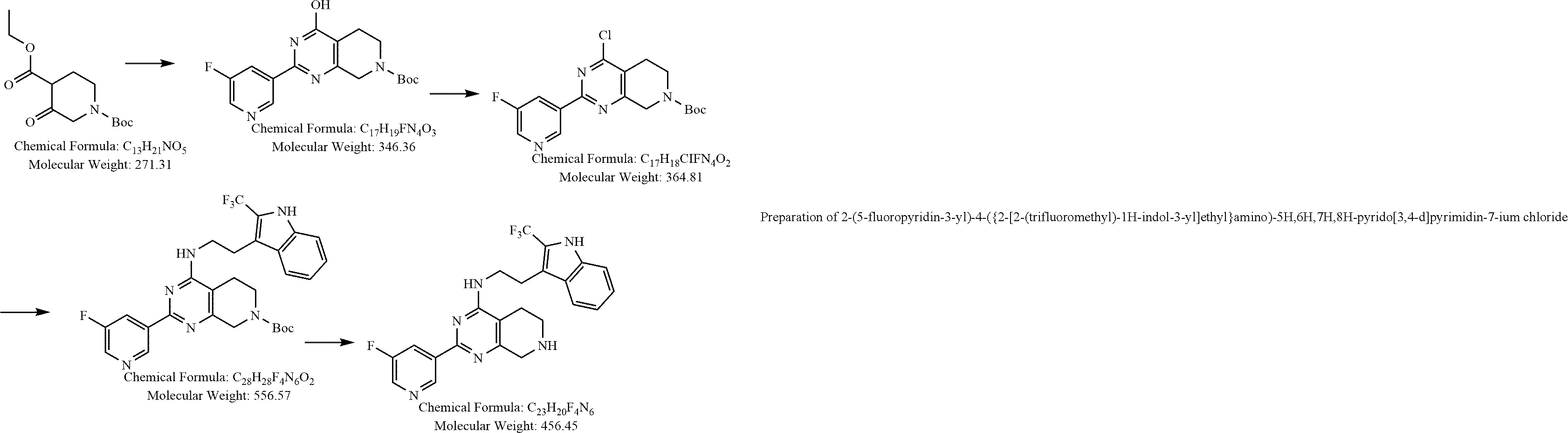

### Step 1 Tert-Butyl 2-(5-Fluoropyridin-3-yl)-4-Oxo-3H,4H,5H,6H,7H,8H-Pyrido[3,4-d]Pyrimidine-7-Carboxylate

A stirred solution of 1-tert-butyl 4-ethyl 3-oxopiperidine-1,4-dicarboxylate (1.05 g, 3.87 mmol, 1.0 eq) in EtOH (10 mL) was treated sequentially with potassium tert-butoxide (1.35 g, 12.0 mmol, 3.10 eq) and 5-fluoropyridine-3-carboximidamide hydrochloride (1.02 g, 5.81 mmol, 1.50 eq), and the resulting mixture was stirred at 60° C. for 16 h. The reaction mixture was then cooled to ambient temperature and treated with water (10 mL), before being evaporated to dryness under reduced pressure. The resulting residue was diluted in water and treated with glacial acetic acid to pH = 7, resulting in the formation of a suspension. The mixture was filtered and the solid washed with hexane and dried under suction to give the desired product as a pale yellow solid (750 mg, 54%).
UPLC-MS (Basic Method, 4 min): rt 1.15 min, m/z 347.1 [M+H]+
19F NMR (376 MHz, DMSO) δ -126.80 Proton decoupled
1 H NMR (400 MHz, DMSO) δ 9.10 (s, 1 H), 8.77 (d, J = 2.7 Hz, 1 H), 8.31 (ddd, J = 10.0, 2.8, 1.8 Hz, 1 H), 4.35 (s, 2 H), 3.57 (t, J = 5.7 Hz, 2 H), 1.44 (s, 9 H). CH2 signal under DMSO solvent peak.

### Step 2 Tert-Butyl 4-Chloro-2-(5-Fluoropyridin-3-yl)-5H,6H,7H,8H-Pyrido[3,4-d]Pyrimidine-7-Carboxylate

A stirred suspension of tert-butyl 2-(5-fluoropyridin-3-yl)-4-oxo-3H,4H,5H,6H,7H,8H-pyrido[3,4-d]pyrimidine-7-carboxylate (750 mg, 2.17 mmol, 1.00 eq) in dichloroethane (20 mL) was treated sequentially with triphenylphosphane (1.13 g, 4.33 mmol, 2.0 eq) and tetrachloromethane (627 mL, 6.49 mmol, 3.0 eq), and the resulting mixture was stirred at 70° C. for 1 h. The reaction mixture was concentrated to dryness to give an oil, which was purified by automated column chromatography (25 g Telos; eluting with a 2:1 v/v mixture of iso-hexane and EtOAc) to give the desired product as a white solid (540 mg, 68%).
UPLC-MS (Basic method, 4 min): rt 2.19 min, m/z 365.1/367.1 [M+H]+ chlorine isotope pattern 19F NMR (376 MHz, DMSO) δ -126.68 Proton decoupled
1 H NMR (400 MHz, DMSO) δ 9.28 (t, J = 1.7 Hz, 1 H), 8.77 (d, J = 2.8 Hz, 1 H), 8.36 (ddd, J = 9.8, 2.9, 1.7 Hz, 1 H), 4.65 (s, 2 H), 3.70 (t, J = 5.8 Hz, 2 H), 2.83 (t, J = 5.8 Hz, 2 H), 1.45 (s, 9 H).

### Step 3 Tert-Butyl 2-(5-Fluoropyridin-3-yl)-4-({2-[2-(Trifluoromethyl)-1H-Indol-3-yl]Ethyl}Amino)-5H,6H,7H,8H-Pyrido[3,4-d]Pyrimidine-7-Carboxylate

A stirred solution of tert-butyl 4-chloro-2-(5-fluoropyridin-3-yl)-5H,6H,7H,8H-pyrido[3,4-d]pyrimidine-7-carboxylate) (50 mg, 0.14 mmol, 1.0 eq) in IPA (1.7 mL) was treated sequentially with 2-[2-(trifluoromethyl)-1H-indol-3-yl]ethan-1-aminium chloride (40 mg, 0.15 mmol, 1.10 eq) and triethylamine (76 mL, 0.55 mmol, 4.0 eq) at 100° C. for 16 h. The reaction formed a suspension, which was filtered at 80° C., and the resulting solid was washed with cold IPA and dried under suction to afford the desired product as a pale pink solid (30 mg, 39%)
UPLC-MS (Basic Method, 4 min): rt 2.32 min, m/z 557.3 [M+H]+
19F NMR (376 MHz, DMSO) δ -56.40, -127.86 Proton decoupled
1 H NMR (400 MHz, DMSO) δ 11.89 (s, 1 H), 9.26 (d, J = 1.8 Hz, 1 H), 8.66 (d, J = 2.9 Hz, 1 H), 8.22 (d, J = 10.1 Hz, 1 H), 7.74 (d, J = 8.1 Hz, 1 H), 7.40 (d, J = 8.3 Hz, 2 H), 7.26 (t, J = 7.6 Hz, 1 H), 7.10 (t, J = 7.5 Hz, 1 H), 4.38 (s, 2 H), 3.80 – 3.74 (m, 2 H), 3.62 (s, 2 H), 3.18 (d, J = 7.4 Hz, 2 H), 2.39 (s, 2 H), 1.44 (s, 9 H).

### Step 4 2-(5-Fluoropyridin-3-yl)-4-({2-[2-(Trifluoromethyl)-1H-Indol-3-yl]Ethyl}Amino)-5H,6H,7H,8H-Pyrido[3,4-d]Pyrimidin-7-ium Chloride

tert-Butyl 2-(5-fluoropyridin-3-yl)-4-({2-[2-(trifluoromethyl)-1H-indol-3-yl]ethyl)amino)-5H,6H,7H,8H-pyrido[3,4-d]pyrimidine-7-carboxylate (30 mg, 0.054 mmol, 1.0 eq) was treated with 4 M HCl in dioxane (1 mL), and the resulting mixture was stirred for 2 h. A suspension formed, which was collected by filtration. The solid was washed with ether and dried under suction to afford the desired product as solid (26 mg, 96%)
UPLC-MS (Basic Method, 4 min): rt 1.72 min, m/z 457.2 [M+H]+
19F NMR (376 MHz, DMSO) δ -56.35, -127.60 Proton decoupled
1 H NMR (400 MHz, DMSO) δ 11.96 (s, 1 H), 9.45 (s, 2 H), 9.29 (t, J = 1.7 Hz, 1 H), 8.71 (d, J = 2.9 Hz, 1 H), 8.28 (ddd, J = 10.0, 2.9, 1.6 Hz, 1 H), 7.77 (d, J = 8.0 Hz, 1 H), 7.67 (t, J = 6.0 Hz, 1 H), 7.43 (dt, J = 8.3, 0.9 Hz, 1 H), 7.29 (ddd, J = 8.2, 7.0, 1.1 Hz, 1 H), 7.13 (ddd, J = 8.0, 7.0, 1.0 Hz, 1 H), 4.17 (s, 2 H), 3.78 (q, J = 6.8 Hz, 2 H), 3.45 (d, J = 6.5 Hz, 2 H), 3.18 (t, J = 7.5 Hz, 2 H), 2.64 (q, J = 7.3, 6.2 Hz, 2 H)

### Biological Activity

Examples were tested in selected biological assays two or more times. Data are reported as the arithmetic mean of the pIC₅₀ (-log₁₀IC₅₀) values, where IC₅₀ is defined as the concentration of compound producing a 50% inhibition of the agonist response. In the case of the U937 assay, VAF347 was used as the agonist; in the peripheral blood mononuclear cell (PBMC) assay the effects of the test compounds are assessed against endogenously produced AhR agonists generated following PBMC activation.

The in vitro activity of the compounds of the present invention was assessed in the following assays:

### In Vitro Assay 1: AhR Antagonism in U937 Cells (Promega P450-Glo™ Assay)

AhR antagonism was assessed in U937 cells (myeloid lineage cell line derived from a human histiocytic lymphoma). Ligand binds the AhR in the cytoplasm, and the AhR-ligand complex translocates to the nucleus and forms a heterodimer with AhR nuclear translocator (Arnt). This complex binds the xenobiotic response element (XRE) in the 5′ upstream region of the CYP1A1 promoter, enhancing CYP1A1 expression. CYP1A1 activity is subsequently determined by assessing the conversion of Luciferin-CEE to luciferin, which in turn reacts with luciferase to produce light. The amount of light produced is directly proportional to cytochrome P450 activity.

U937 cells in Ultraculture serum free media (Lonza) were plated at 100,000 cells per well in a round bottom 96 well tissue culture plate. Seven concentrations of test compound (final [DMSO] 1%) were added and incubated for 10 minutes before the addition of 4.5 nM VAF347. The plates were then placed in an incubator at 37° C., ≥ 85% humidity, 5% CO₂ for 24 hrs. After aspiration of the supernatant the CYP1A1 substrate Luciferin-CEE ([Final] 83 µM) was added and incubated for 3 hrs before the reaction was stopped by adding luciferin detection reagent and luminescence was read after 20 minutes.

### In Vitro Assay 2: AhR Antagonism in Human Peripheral Blood Mononuclear Cells (PBMC_{S}) -Inhibition of Interleukin-22 (IL-22) Release

PBMCs were isolated from human peripheral blood using Lymphoprep™ and diluted to 1 × 10⁶ cells per ml in RPMI media containing 10% foetal bovine serum, 1% penicillin-streptomycin and 1% non-essential amino acids. PBMCs were subsequently activated with 1 µl per 100,000 cells of a CD3/CD28 agonist mixture (human T Cell TransAct™(Miltenyi Biotec)) and then plated at 100,000 cells per well in a round bottom 96 well tissue culture plate. One hour after stimulation seven concentrations of each test compound or vehicle (final [DMSO] 0.2%) were added. The plates were then placed in an incubator at 37° C., ≥ 85% humidity, 5% CO₂ for 72 hrs before the media was removed and stored at -20° C. until cytokine analysis. IL-22 was measured using human IL-22 DuoSet ELISA (R&D systems) according to the manufacturer’s instructions. The results are shown in Table 2:

| Example No: | U937 Assay (pIC₅₀) | PBMC Assay (pIC₅₀) |
|---|---|---|
| 1 | 6.9 | 6.9 |
| 5 | 6.7 | 6.4 |
| 9 | 7.7 | 6.1 |
| 10 | 7.6 | 6.1 |
| 14 | 7.0 | 6.2 |
| 15 | 6.8 | 6.2 |
| 16 | 6.5 | 6.7 |
| 17 | 7.3 | 7.6 |
| 18 | 7.7 | 6.5 |

### In Vitro Assay: CYP1A1 Inhibition Assay

The direct CYP1A1 inhibitory activity of test compounds was also assessed using the Promega P450-Glo™ assay system. Seven concentrations of test compound were added to a ½ area white 96 well plate. Cypex CYP1A1 bactosomes ([final] 0.5 pmol) and CYP1A1 substrate Luciferin-CEE ([final] 30 µM) were prepared in 0.1 M potassium phosphate buffer and incubated with test compounds at 37° C. for 5 minutes. 0.2 mM NADPH was then added to the plates and incubated at 37° C., for 10 minutes. The reaction was stopped by adding luciferin detection reagent and luminescence was read after 20 minutes.

1-19. (canceled)

20. A compound of formula (I) 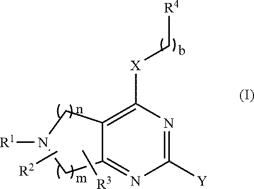
wherein:
Y is a 5 or 6 membered ring optionally comprising 1, 2, or 3 heteroatoms selected from N, O and S, substituted with R⁵ and R⁶;
R¹ is H, C₁₋₃ alkyl, (—CH₂)pCN, -COC₁₋₃ alkyl, —CO(CH₂)qNR⁷R⁸, -SO₂C₁₋₃ alkyl, —SO₂NR⁷R⁸, —(CH₂)qPh, —C(O)Z;
R² is H or C₁₋₃ alkyl;
R³ is H or C₁₋₃ alkyl;
R⁴ is a 9 or 10 membered heteroaryl with at least one heteroatom selected from N, O or S (such as Indol-3-yl or Benzimidazol-2-yl), with substituents R⁹ and R¹⁰;
R⁵ is located beta to atom which is bonded to the remainder of the molecule and is selected from hydroxy, halogen (such as F, Cl), CN, C₁₋₃ alkyl, C₁₋₃ alkoxy (such as OMe), C₁₋₂ haloalkyl (such as CF₃), C₁₋₃ alkyl(OH), —CO(CH₂)qNR⁷R⁸, -SO₂C₁₋₃alkyl, —SO₂ NR⁷R⁸,
R⁶ is H, hydroxy, halogen (such as F, Cl), CN, C₁₋₃ alkyl, —CO(CH₂)qNR⁷R⁸, —SO₂C₁₋₃ alkyl, —SO₂ NR⁷R⁸,
R⁷ is H or C₁₋₃ alkyl, such as —CH₃;
R⁸ is H or C₁₋₃ alkyl, such as —CH₃;
R⁹ is H, hydroxy, halogen (such as F, Cl), CN, C₁₋₃ alkyl, C₁₋₃ alkoxy (such as OMe), C₁₋₂ haloalkyl (such as CF₃), C₁₋₃ alkyl(OH),—CO(CH₂)q NR⁷R⁸, -SO₂C₁₋₃alkyl, or —SO₂ NR⁷R⁸,
R¹⁰ is H, hydroxy, halogen (such as F, Cl), CN, C₁₋₃ alkyl, —CO(CH₂)q NR⁷R⁸, —SO₂C₁₋₃ alkyl, or —SO₂ NR⁷R⁸,
R¹¹ is H or C₁₋₃ alkyl (such as —CH₃);
X is CH₂, S, —SO₂, NR¹¹ or O;
Z is a 5 or 6 membered heteroaryl with at least one heteroatom selected from N, O and S, for example 1 or 2 nitrogens, wherein said heteroaryl optionally bears one or two substituents selected from hydroxy, halogen (such as F, Cl), CN, C₁₋₃ alkyl;
b is 0, 1, 2 or 3 (for example 0 or 2);
p is an integer 1, 2 or 3 (such as 1);
q is 0, 1, 2 or 3 (such as 0 or 1),
wherein:
n is an integer 1 and m is an integer 1 or 2; or
m is an integer 1 and n is an integer 2;
or a pharmaceutically acceptable salt thereof
with the proviso that when X is NR¹¹ or O and b is 1 or 2 then R⁵ or R⁹ is selected from C₁₋₃ alkoxy (such as OMe), C₁₋₂ haloalkyl (such as CF₃), and C₁₋₃ alkyl(OH).

21. A compound of formula (IA) 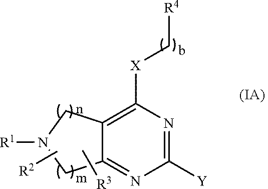
wherein:
Y is a 5 or 6 membered ring comprising 1, 2, or 3 heteroatoms selected from N, O and S, substituted with R⁵ and R⁶;
R¹ is H, C₁₋₃ alkyl, (—CH₂)pCN, -COC₁₋₃ alkyl, —CO(CH₂)qNR⁷R⁸, -SO₂C₁₋₃ alkyl, —SO₂NR⁷R⁸, —(CH₂)qPh, —C(O)Z;
R² is H or C₁₋₃ alkyl;
R³ is H or C₁₋₃ alkyl;
R⁴ is a 9 or 10 membered heteroaryl with at least one heteroatom selected from N, O or S (such as Indol-3-yl or Benzimidazol-2-yl), with substituents R⁹ and R¹⁰;
R⁵ is hydroxy, halogen (such as F, Cl), CN, C₁₋₃ alkyl, C₁₋₃ alkoxy (such as OMe), C₁₋₂ haloalkyl (such as CF₃), C₁₋₃ alkyl(OH), —CO(CH₂)qNR⁷R⁸, -SO₂C₁₋₃alkyl, —SO₂ NR⁷R⁸,
R⁶ is H, hydroxy, halogen (such as F, Cl), CN, C₁₋₃ alkyl, —CO(CH₂)qNR⁷R⁸, -SO₂C₁₋₃ alkyl, —SO₂ NR⁷R⁸,
R⁷ is H or C₁₋₃ alkyl, such as —CH₃;
R⁸ is H or C₁₋₃ alkyl, such as —CH₃;
R⁹ is H, hydroxy, halogen (such as F, Cl), CN, C₁₋₃ alkyl, C₁₋₃ alkoxy (such as OMe), C₁₋₂ haloalkyl (such as CF₃), C₁₋₃ alkyl(OH),—CO(CH₂)q NR⁷R⁸, —SO₂C₁₋₃alkyl, or —SO₂ NR⁷R⁸,
R¹⁰ is H, hydroxy, halogen (such as F, Cl), CN, C₁₋₃ alkyl, —CO(CH₂)q NR⁷R⁸, -SO₂C₁₋₃ alkyl, or —SO₂ NR⁷R⁸,
R¹¹ is H or C₁₋₃ alkyl (such as —CH₃);
X is CH₂, S, —SO₂, NR¹¹ or O;
Z is a 5 or 6 membered heteroaryl with at least one heteroatom selected from N, O and S, for example 1 or 2 nitrogens, wherein said heteroaryl optionally bears one or two substituents selected from hydroxy, halogen (such as F, Cl), CN, C₁₋₃ alkyl;
b is 0, 1, 2 or 3 (for example 0 or 2);
n is an integer 1 or 2;
m is an integer 1 or 2;
p is an integer 1, 2 or 3 (such as 1);
q is 1, 2 or 3 (such as 1),
or a pharmaceutically acceptable salt thereof
with the proviso that when X is NR¹¹ or O and b is 1 or 2 then R⁵ or R⁹ is selected from C₁₋₃ alkoxy (such as OMe), C₁₋₂ haloalkyl (such as CF₃), and C₁₋₃ alkyl(OH).

22. A compound of formula (I) according to claim 20, wherein Y is a 5 or 6 membered nitrogen containing ring.

23. A compound of formula (I) according to claim 22, wherein the ring is aromatic.

24. A compound of formula (I) according to claim 23, wherein the ring is pyrimidine or pyridine.

25. A compound of formula (I) according to claim 24, wherein R⁵ is located at position 5.

26. A compound of formula (II) 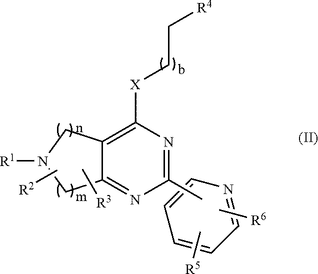
wherein X, R¹, R², R³, R⁴, R⁵, R⁶, b, m and n are defined above for compounds of formula (IA) or a pharmaceutically acceptable salt thereof.

27. A compound of formula (III): 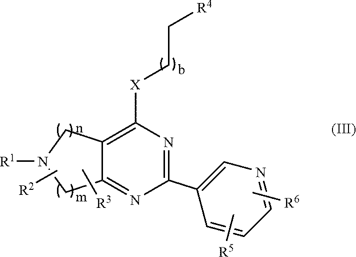
wherein X, R¹, R², R³, R⁴, R⁵, R⁶, b, m and n are defined above for compounds of formula (IA) or a pharmaceutically acceptable salt thereof.

28. A compound according to claim 20, wherein n is 2.

29. A compound according to claim 20, wherein n is 1.

30. A compound according to claim 20, wherein m is 2.

31. A compound according to claim 20, wherein m is 1.

32. A compound according to claim 20, of formula (IV): 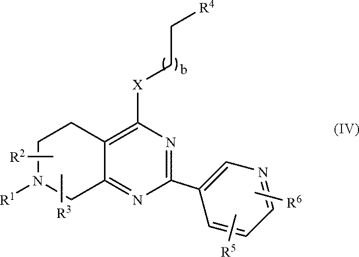
wherein X, R¹, R², R³, R⁴, R⁵, R⁶ and b, are defined above for compounds of formula (IA) or a pharmaceutically acceptable salt thereof.

33. A compound according to claim 20, of formula (V): 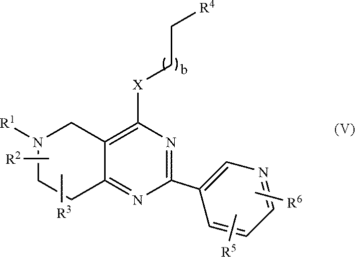
wherein X, R¹, R², R³, R⁴, R⁵, R⁶ and b, are defined above for compounds of formula (IA) or a pharmaceutically acceptable salt thereof.

34. A compound according to claim 20, of formula (VI): 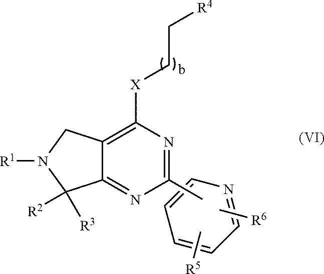
wherein X, R¹, R², R³, R⁴, R⁵, R⁶ and b, are defined above for compounds of formula (I) or a pharmaceutically acceptable salt thereof.

35. A compound according to claim 21 of formula (VII): 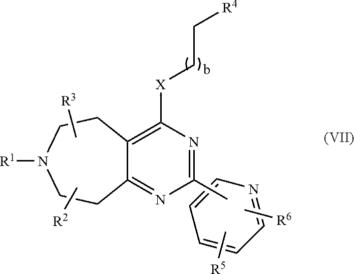
wherein X, R¹, R², R³, R⁴, R⁵, R⁶ and b, are defined above for compounds of formula (IA) or a pharmaceutically acceptable salt thereof.

36. A pharmaceutical composition comprising a compound according to claim 20, and a pharmaceutically acceptable diluent or carrier.

37. A method of treatment comprising administering a therapeutically effective amount of a compound according to claim 20 to a patient in need thereof, for example for the treatment of cancer.
